(19) United States
(12) Patent Application Publication
(10) Pub. No.: US 2017/0035854 A1
(43) Pub. Date: Feb. 9, 2017

(54) CONTROLLED OVARIAN HYPERSTIMULATION WITH IMPROVED RECOMBINANT HUMAN FOLLICLE-STIMULATING HORMONE

(71) Applicant: Glycotope GmbH, Berlin (DE)

(72) Inventors: Steffen GOLETZ, Berlin (DE); Lars STÖCKL, Berlin (DE)

(21) Appl. No.: 15/303,703

(22) PCT Filed: Apr. 17, 2015
(86) PCT No.: PCT/EP2015/058352

§ 371 Date: Oct. 12, 2016

Related U.S. Application Data
(60) Provisional application No. 61/981,621, filed on Apr. 18, 2014.

(51) Int. Cl. A61K 38/24; C12N 5/075; A61B 17/43; A61K 38/09

(52) U.S. Cl. CPC A61K 38/24; A61K 38/09; C12N 5/0609; A61B 17/43

(57) ABSTRACT

The present invention pertains to methods for controlled ovarian hyperstimulation in a female subject using improved recombinant human follicle-stimulating hormone (rhFSH). The methods result in a high number of fertilizable oocytes even at low amounts of FSH administered to the female subject.

### FIELD OF THE INVENTION

The present invention pertains to the field of infertility treatment. In particular, methods for controlled ovarian hyperstimulation with improved recombinant human follicle-stimulating hormone (rhFSH) are provided. The methods described herein result in a higher number of fertilizable oocytes in the treated women using a lower amount of FSH than in conventional treatments.

### BACKGROUND OF THE INVENTION

Gonadotropins are a group of protein hormones which regulate gonadal function in the male and female and thereby play an important role in human fertility. They are secreted by gonadotrope cells of the pituitary gland of vertebrates after stimulation by the gonadotropin-releasing hormone (GnRH). Gonadotropins are heterodimeric glycoproteins including follicle stimulating hormone (FSH), luteinizing hormone (LH) and chorionic gonadotropin (CG). The gonadotropins share identical alpha-subunits but comprise different beta-subunits which ensure receptor binding specificity.

FSH comprises a 92 amino acid alpha-subunit and a 111 amino acid beta-subunit which confers specific binding to the FSH receptor. Both subunits of the natural protein are modified by glycosylation. The alpha-subunit is naturally glycosylated at Asn52 and Asn78 and the beta-subunit at Asn7 and Asn24. Both subunits are produced in the cells as precursor proteins and then processed and secreted. FSH regulates the development, growth, pubertal maturation, and reproductive processes of the body. In particular, it stimulates the maturation of germ cells and thus is involved in spermatogenesis and folliculogenesis.

Folliculogenesis is induced by FSH, for example, by binding of FSH to FSH receptors on the surface of granulosa cells. FSH receptors are G protein-coupled receptors which activate the coupled G protein upon binding of FSH. The G protein in turn activates adenylyl cyclase, resulting in the production of cAMP, a second messenger molecule. The increasing cAMP concentration in the cell activates several downstream targets, in particular cAMP dependent protein kinases, which then lead to the synthesis of progesterone and estradiol. Then progesterone and estradiol is secreted by the granulosa cells, inducing folliculogenesis. Upon stimulation of the granulosa cells by FSH, they also release inhibin-B which forms a negative feedback loop, inhibiting the production and secretion of FSH in the pituitary gland. Inhibin-B was shown to be a good surrogate marker for the ovarian stimulation by FSH.

FSH is widely used in the treatment of infertility, either alone or in combination with other agents, in particular LH. In the art, generally FSH purified from post-menopausal human urine (urinary FSH) or FSH recombinantly produced by Chinese hamster ovary (CHO) cells has been used for human treatment. Recombinant FSH obtained from CHO cells is for example disclosed in WO 03/035686 A2. However, there is considerable heterogeneity associated with FSH preparations due to different isoforms present. Individual FSH isoforms exhibit identical amino acid sequences but differ in the extent and nature of their glycosylation. Particular isoforms are characterized by heterogeneity of the carbohydrate branch structures and differing amounts of sialic acid (a negatively charged terminal monosaccharide unit) incorporation, both of which influence the specific bioactivity of the isoform. Thus, the glycosylation pattern of the FSH has a significant influence on its biological activity.

However, urinary FSH from different donors and different preparations can significantly vary in its carbohydrate structures, resulting in a high batch-to-batch variation. There are also safety concerns regarding the presence of viruses in the urinary products. Furthermore, FSH obtained from CHO cells exhibits a glycosylation pattern specific for these hamster cells which is not identical to human glycosylation patterns. These differences result in varying biological activities and adverse effects of the obtained FSH and thus, of the pharmaceutical preparations which are to be administered to the patient. Adverse side effects accompanying FSH treatment include, for example, ovarian cyst formation, ovarian hyperstimulation syndrome (OHSS), multiple pregnancy, hot flushes, feeling down or irritable, headaches, restlessness, nausea, vomiting, shortage of breath, abdominal bloating due to accumulation of fluids, abdominal pain and enlargement of the ovaries.

Recently, improved FSH obtained from human myeloid leukemia cells has been developed which shows remarkable biological and pharmaceutical properties (see WO 2012/017058 A1). This FSH preparation is highly active and activates secretion of progesterone and estradiol in granulosa cells even at low concentration. Furthermore, this FSH has a fully human glycosylation pattern which is stably produced in the human cell line without any safety concerns.

Besides supporting natural fertilization, FSH treatment is used for inducing the development of multiple ovarian follicles. With such a treatment cycle of controlled ovarian hyperstimulation, several mature oocytes can be obtained from a female patient. After retrieval of the oocytes, they are fertilized in vitro and returned into the female body. However, for such assisted reproductive technologies (ART), high concentration FSH administrations are necessary which bear the risk of adverse side effects. In particular, ovulary hyperstimulation syndrome is a common risk associated with infertility treatments. Reducing the amount of FSH administered, however, also reduces the number of oocytes obtained per treatment cycle and hence, the chance of a successful fertilization and nidation of an embryo in the uterus.

Therefore, there is the need in the art for improved FSH treatments for controlled ovarian hyperstimulation which lead to high numbers of induced oocytes at low amounts of administered FSH. In view of this, it is one object of the present invention to provide improved infertility treatments.

### SUMMARY OF THE INVENTION

The present inventors have found that improved FSH preparations having an optimized glycosylation pattern are able to induce a superior follicle growth and a high number of mature oocytes, even when using dosage regimens with a low overall amount of FSH. In particular, it was demonstrated that the improved FSH preparations stimulate the development of multiple oocytes in a female subject at dosage regiments wherein only half of the amount of FSH is administered compared to the commonly used dosage regiments with commercially available FSH preparations (see Example 2). Indeed, the number of induced follicles having a size of at least 12 mm, the number of cumulus-oocyte complexes (COCs) retrieved from the patients, the number of fertilizable metaphase II oocytes retrieved from the patients and the number of successfully fertilized oocytes (two pronuclei (2PN) oocytes) are increased for patients receiving the recombinant FSH preparation described herein when compared to patients receiving a higher amount of Gonal-f, a commercially available FSH preparation obtained from CHO cells. Furthermore, also the quality of the induced follicles was superior for the improved FSH preparations as a higher percentage amount of the induced follicles can successfully be fertilized compared to the follicles induced by Gonal-f. Currently available techniques of cryopreservation of surplus oocytes (2PN) or embryos enable the fertility clinics to perform subsequent embryo transfers in case that the first transfer did not lead to pregnancy by thawing and transferring these surplus embryos without another FSH stimulation cycle. Thus, the higher number of fertilized oocytes directly results in an increased number of transferred embryos (per stimulation cycle) and a higher chance for nidation of an embryo in the uterus.

Furthermore, it was found that the administration of FSH every second day or less frequently is also possible and leads to good therapeutic results. These findings were highly unexpected since longer administration intervals result in unwanted fluctuations in the FSH serum level which were considered to have a negative impact on follicular growth and may result in growth arrest of the follicles or even decline of the follicle size. The present inventors indeed observed significant fluctuations in the serum level of FSH in the patients when administering the FSH every second day or less frequently (see Example 3 and FIG. 4). These fluctuations were expected since the improved FSH used according to the present invention has a rather low circulation half-life which is similar to the commonly used recombinant FSH from CHO cells and even lower than the half-life of urinary FSH (see Example 3 and FIG. 12). However, in contrast to the assumption in the prior art, these fluctuations did not arrest the follicle growth. Rather, it was found that follicle growth was even enhanced compared to daily administrations of equal overall amounts of the same improved FSH or to daily administrations of even double overall amounts of the commonly used FSH (see Example 3 and FIGS. 1 to 9). These unexpected superior therapeutic results were obtained using unmodified FSH preparations having a human glycosylation pattern as described herein. No artificial modifications such as genetically engineered FSH or FSH conjugates have to be used.

In view of the above findings, the present invention provides a method for controlled ovarian hyperstimulation for stimulating the development of multiple ovarian follicles in a female subject, wherein a recombinant FSH preparation is administered to the female subject, wherein the recombinant FSH preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation.

In a first aspect, the recombinant FSH preparation is administered to the female subject using a dosage regimen wherein the single doses sum up to an average amount of from about 35 to about 250 IU FSH per day. When there are multiple follicles with a mean diameter equal to or greater than 12 mm and/or when there is at least one follicle with a diameter of at least 17 mm, ovulation is triggered. Then multiple oocytes are obtained from the female subject, wherein on average at least 5 oocytes per female subject are obtained and/or at least 5 oocytes from the female subject are obtained.

In a second aspect, a dosage regimen is used wherein the recombinant FSH preparation is administered in an amount in IU which is 80% or less of the amount recommended for recombinant FSH preparations produced by CHO cells in the same therapeutic situation. When there are multiple follicles with a mean diameter equal to or greater than 12 mm and/or when there is at least one follicle with a diameter of at least 17 mm, ovulation is triggered. Then multiple oocytes are obtained from the female subject, wherein on average at least 5% more oocytes per female subject are obtained compared to a similar treatment with the amount recommended for recombinant FSH preparations produced by CHO cells in the same therapeutic situation.

Furthermore, it was found that follicles grown due to stimulation with the recombinant FSH preparation as described herein maintain their size in the human body for a significant time interval after termination of the FSH administration (see Example 3 and FIG. 11). In particular, the follicles essentially remain at their maximum size for several days. In contrast, follicles grown due to stimulation with conventional FSH rapidly regress one or two days after reaching their maximum size. Because of this regression, in the common treatments final maturation and ovulation had to be triggered in a small time interval, generally one day and at most 36 hours after termination of the FSH administration. With the advantageous recombinant FSH preparation as described herein, final maturation and ovulation can be triggered until up to 6 days after termination of the FSH administration. This provides the infertility treatment with a much greater flexibility. In particular, scheduling, planning and organizing the subsequent steps such as triggering ovulation and oocyte retrieval is greatly improved and simplified using the recombinant FSH preparation as described herein.

In view of this, the present invention provides in a third aspect a method for stimulating follicle maturation in a female subject, comprising inducing or enhancing follicle growth in a female subject by administering a recombinant FSH preparation, and subsequently triggering ovulation which is commenced at least 48 h after termination of the administration of the recombinant FSH preparation, wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DETAILED DESCRIPTION OF THE INVENTION

The present invention is based on the finding that the recombinant FSH preparation described herein having an improved glycosylation pattern is capable of inducing the growth of higher numbers of large follicles in female subjects, even at lower amounts of FSH administered to the subjects, when compared to conventional FSH preparations such as CHO-derived FSH, for example Gonal-f. In particular, with half the amount of FSH, the recombinant FSH preparation as described herein leads to similar or even better results in follicle growth compared to FSH produced in CHO cells used at normal amounts.

In view of these findings, the present invention provides in a first aspect a method for controlled ovarian hyperstimulation for stimulating the development of multiple ovarian follicles in a female subject, comprising:
(a) administering to a female subject a recombinant FSH preparation using a dosage regimen wherein the single doses sum up to an average amount of from about 35 to about 250 IU FSH per day;
(b) triggering ovulation when there are multiple follicles with a mean diameter equal to or greater than 12 mm and/or when there is at least one follicle with a diameter of at least 17 mm;
(c) obtaining multiple oocytes from the female subject, wherein on average at least 5 oocytes per female subject are obtained and/or at least 5 oocytes from the female subject are obtained;

wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation.

In a second aspect, the present invention provides a method for controlled ovarian hyperstimulation for stimulating the development of multiple ovarian follicles in a female subject, comprising
(a) administering to a female subject a recombinant FSH preparation using a dosage regimen wherein the recombinant FSH preparation is administered in an amount in IU which is 80% or less of the amount recommended for recombinant FSH preparations produced by CHO cells in the same therapeutic situation;
(b) triggering ovulation when there are multiple follicles with a mean diameter equal to or greater than 12 mm and/or when there is at least one follicle with a diameter of at least 17 mm;
(c) obtaining multiple oocytes from the female subject, wherein on average at least 5% more oocytes per female subject are obtained compared to a similar treatment with the amount recommended for recombinant FSH preparations produced by CHO cells in the same therapeutic situation;

wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation.

In a third aspect, the present invention provides a method for stimulating follicle maturation in a female subject, comprising
(a) inducing or enhancing follicle growth in a female subject by administering a recombinant FSH preparation; and
(b) subsequently triggering ovulation;

wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation;

wherein triggering ovulation in step (b) is commenced at least 48 h after termination of the administration of the recombinant FSH preparation in step (a).

The Recombinant FSH

A “FSH preparation” may be any composition or substance comprising or consisting of FSH. It may be in solid or fluid form and may comprise further ingredients in addition to FSH. In particular, a FSH preparation may be a solution comprising FSH and a suitable solvent such as water and/or alcohol, or a powder obtained, for example, after lyophilization of a solution containing FSH. Suitable examples of a FSH preparation are compositions obtained after expression of FSH in cells, in particular after purification of the FSH, or pharmaceutical compositions comprising FSH. A FSH preparation may contain, in addition to FSH, for example solvents, diluents, excipients, stabilizers, preservatives, salts, adjuvants and/or surfactants. The terms “FSH preparation” is used herein in particular in the meaning of a “composition comprising FSH”. These terms are preferably used synonymously herein.

The term “FSH” as used herein refers to follicle-stimulating hormone, a gonadotropin. FSH is a glycoprotein comprised of two subunits, labeled alpha and beta subunits. Preferably, the FSH is human FSH, in particular human FSH composed of an alpha subunit having the amino acid sequence of SEQ ID NO: 1 and an beta subunit having the amino acid sequence of SEQ ID NO: 2. However, one or more, such as 1, 1 or 2, up to 3, up to 5, up to 10 or up to 20, amino acid substitutions, additions and/or deletions may be present in one or both subunits. Preferably, the amino acid sequence of the alpha subunit shares an overall homology or identity of at least 80%, more preferably at least 85%, at least 90%, at least 95% or at least 98% with the amino acid sequence according to SEQ ID NO: 1 over its entire length. Furthermore, the amino acid sequence of the beta subunit preferably shares an overall homology or identity of at least 80%, more preferably at least 85%, at least 90%, at least 95% or at least 98% with the amino acid sequence according to SEQ ID NO: 2 over its entire length. The subunits of the FSH are preferably two separate polypeptide chains, however, the term “FSH” as used herein also encompasses embodiments wherein the two subunits are covalently attached to each other, e.g. by cross-linking agents or a linking polypeptide chain, and embodiments, wherein one or both subunits are further divided into several polypeptide chains. Preferably, the FSH according to the invention is capable of binding to and/or activating the FSH receptor, preferably the human FSH receptor. The term “FSH” as used herein in particular refers to all FSH proteins in a preparation. Thus, the term “FSH” in particular refers to the entirety of all FSH proteins in a FSH preparation or composition.

The FSH according to the present invention is glycosylated, i.e. it is modified by one or more, preferably four, oligosaccharides attached to the polypeptides chains. These oligosaccharides, also named glycans, carbohydrates or carbohydrate structures, may be linear or branched saccharide chains and preferably are complex-type N-linked oligosaccharide chains. Depending on the number of branches the oligosaccharide is termed mono-, bi-, tri- or tetraantennary (or even pentaantennary). A monoantennary oligosaccharide is unbranched, i.e. it has no branching point and comprises only one antenna, while a bi-, tri- or tetraantennary oligosaccharide has one, two or three branching points and hence, two, three or four antennae, respectively. A glycoprotein with a higher antennarity thus has more oligosaccharide endpoints and can carry more functional terminal saccharide units such as, for example, sialic acids. “At least triantennary” as used herein refers to oligosaccharides having an antennarity of at least 3, including triantennary, tetraantennary and pentaantennary oligosaccharides. “At least tetraantennary” as used herein refers to oligosaccharides having an antennarity of at least 4, including tetraantennary and pentaantennary oligosaccharides. With respect to complex-type N-glycans, a bisecting GlcNAc residue preferably is not considered as a branch or antenna and thus, does not add to the antennarity of the FSH. The terms “branch” and “antenna” of a glycan structure are use synonymously herein.

The glycosylation pattern of FSH as referred to herein in particular refers to the overall glycosylation pattern of all FSH proteins in a FSH preparation according to the present invention. In particular, any glycan structures comprised in the FSH protein and thus, attached to the FSH polypeptide chains in the FSH preparation are considered and reflected in the glycosylation pattern.

Preferably, both subunits of the FSH protein comprise one or more carbohydrate structures attached to the polypeptide chain. More preferably, the carbohydrate structures are attached to an asparagine residue of the subunits. In particularly preferred embodiments, the alpha subunit comprises two carbohydrate structures preferably attached to asparagine residues corresponding to Asn52 and Asn78 of the human amino acid sequences of the alpha subunit according to SEQ ID NOs: 1, and/or the beta-subunit comprises two carbohydrate structures preferably attached to asparagine residues corresponding to Asn7 and Asn24 of the human amino acid sequences of the beta subunit according to SEQ ID NOs: 2. In certain embodiments, the alpha subunit comprises not more than two carbohydrate chains and the beta subunit comprises not more than two carbohydrate chains, which are preferably attached to the asparagine residues mentioned above. In this embodiment, no additional glycosylation sites and in particular no artificially introduced glycosylation sites are present in the amino acid sequences of FSH. The carbohydrate part of human FSH is preferably composed of fucose, galactose, mannose, galactosamine, (N-acetyl) glucosamine, and/or sialic acid residues. In particular, the carbohydrate part of human FSH is essentially composed of N-acetyl glucosamine, mannose, galactose, sialic acid, fucose and sulfate residues.

The FSH as used according to the present invention is recombinant, preferably recombinant human FSH. The term “recombinant FSH” refers to FSH which is not naturally produced by a living human or animal body and then obtained from a sample derived therefrom, such as urine, blood or other body liquid, feces or tissue of the human or animal body. Preferably, recombinant FSH is obtained from cells which have been biotechnologically engineered, in particular cells which have been transformed or transfected with a nucleic acid encoding FSH or the alpha or beta subunits of FSH. According to preferred embodiments, recombinant FSH is obtained from human host cells comprising an exogenous nucleic acid encoding FSH. Respective exogenous nucleic acids can be introduced e.g. by using one or more expression vectors, which can be introduced into the host cell e.g. via transfection. Respective methods for recombinantly producing proteins and FSH are well known in the prior art and thus, need no further description. Furthermore, suitable host cells for recombinantly producing FSH are described herein.

The FSH preparation according to the invention is characterized by its glycosylation pattern which also distinguishes the present FSH preparation from commonly used FSH preparations, in particular those produced in CHO cells or obtained from human urine.

In preferred embodiments, the recombinant FSH in the preparation has a relative amount of glycans carrying bisecting N-acetylglucosamine (bisGlcNAc) of at least 20% of all glycans attached to the FSH in the preparation. The relative amount of glycans carrying bisGlcNAc is preferably at least 23%, at least 25%, at least 27% or at least 30%. More preferably, it is in the range of from about 20% to about 50%, in particular in the range of from about 25% to about 40% or in the range of from about 28% to about 35%.

A “relative amount of glycans” according to the invention refers to a specific percentage or percentage range of the glycans attached to the FSH glycoproteins of a FSH preparation. In particular, the relative amount of glycans refers to a specific percentage or percentage range of all glycans comprised in the FSH proteins and thus, attached to the FSH polypeptide chains in a FSH preparation. 100% of the glycans refers to all glycans attached to the FSH glycoproteins of the FSH preparation. For example, a relative amount of glycans carrying bisecting GlcNAc of 60% refers to a FSH preparation wherein 60% of all glycans comprised in the FSH proteins and thus, attached to the FSH polypeptide chains in said FSH preparation comprise a bisecting GlcNAc residue while 40% of all glycans comprised in the FSH proteins and thus, attached to the FSH polypeptide chains in said FSH preparation do not comprise a bisecting GlcNAc residue.

In certain embodiments, the recombinant FSH in the preparation has a relative amount of sulfated glycans of at least 2% of all glycans attached to the FSH in the preparation. Preferably, the relative amount of glycans carrying a sulfate group (sulfated glycans) is at least 2.5%, at least 3%, at least 4%, at least 5%, or at least 6%, more preferably at least 7% or at least 8%. According to one embodiment, the relative amount of glycans carrying a sulfate group does not exceed 50%, preferably it is 40% or less, 35% or less, 30% or less, 25% or less or 20% or less.

The glycosylation pattern of the recombinant FSH in the preparation may comprise a relative amount of glycans carrying one or more sialic acid residues of at least 80% of all glycans attached to the FSH in the preparation. The relative amount of glycans carrying one or more sialic acid residues is preferably at least 83%, at least 85% or at least 88%, and more preferably, the relative amount of glycans carrying one or more sialic acid residues is in the range of from about 85% to about 98% or in the range of from about 88% to about 95%, most preferably about 90%. The term “sialic acid” in particular refers to any N- or O-substituted derivatives of neuraminic acid. It may refer to both 5-N-acetylneuraminic acid (NeuNAc) and 5-N-glycolylneuraminic acid (NeuGc), but preferably only refers to 5-N-acetylneuraminic acid. The sialic acid, in particular the 5-N-acetylneuraminic acid preferably is attached to a carbohydrate chain via a 2,3- or 2,6-linkage. Preferably, in the FSH preparations described herein both 2,3- as well as 2,6-coupled sialic acids are present.

In preferred embodiments, the glycosylation pattern of the recombinant FSH in the preparation has a relative amount of 2,6-coupled sialic acid of at least 40% of all sialic acid residues attached to the FSH in the preparation. A “relative amount of 2,6-coupled sialic acid” refers to a specific percentage or percentage range of the total amount of sialic acids being 2,6-coupled sialic acids. A relative amount of 2,6-coupled sialic acid of 100% thus means that all sialic acids that are found on glycans carrying one or more sialic acid residues are 2,6-coupled sialic acids. For example, a relative amount of 2,6-coupled sialic acids of 60% refers to a FSH preparation wherein 60% of all sialic acids comprised in the FSH proteins and thus, attached to the oligosaccharide chains of the FSH proteins in said FSH preparation are attached via a 2,6-linkage while 40% of all sialic acids comprised in the FSH proteins and thus, attached to the oligosaccharide chains of the FSH proteins in said FSH preparation are not attached via a 2,6-linkage, but for example via a 2,3-linkage or a 2,8-linkage. Preferably, the relative amount of 2,6-coupled sialic acid in the recombinant FSH in the preparation is at least 45%, at least 50%, at least 53%, at least 55%, at least 60% or at least 65%, in particular in the range of about 40% to about 99%, preferably about 40% to about 80%, about 50% to about 75% or about 53% to about 70%. Preferably, the ratio of 2,6-coupled sialic acid to 2,3-coupled sialic acid is in the range of from about 2:3 to about 10:1, more preferably from about 2:3 to about 5:1 or from about 1:1 to about 2:1, most preferably from about 1:1 to about 3:2. In preferred embodiments, the relative amount of 2,6-coupled sialic acids exceeds that of 2,3-coupled sialic acids.

The degree of sialylation of FSH may also be expressed as Z-number. The Z-number indicates the relative negative charge of the glycan structures of a glycoprotein. The Z-number is calculated by the formula:

Z=A1%*1+A2%*2+A3%*3+A4%*4

wherein A1% is the percentage of glycans with a charge of −1, A2% is the percentage of glycans with a charge of −2, A3% is the percentage of glycans with a charge of −3, and A4% is the percentage of glycans with a charge of −4. These percentages are calculated with respect to all glycans attached to the FSH, including charged as well as uncharged glycans. The charge of the glycans may be provided by any charged monosaccharide units or substituents comprised in the glycan, in particular by sialic acid residues and/or sulfate groups and/or phosphate groups. Since the charge of the glycans of FSH is generally only determined by their sialic acid residues and FSH generally has four glycan structures, the Z-number is an indication for the amount of sialic acids on the FSH or the acidity of the FSH. However, when the FSH also comprises a significant amount of sulfated glycans, the Z-number is an indication for the combined amounts of sialic acids and sulfate groups.

The recombinant FSH in the composition preferably has a Z-number of at least 200. The Z-number is preferably at least 210, more preferably at least 215 or at least 220. A higher Z-number is for example obtainable by enriching the FSH preparation obtained from the host cells for acidic and/or negatively charged FSH proteins.

In certain embodiments, the glycosylation pattern of the recombinant FSH in the preparation may comprise a relative amount of at least tetraantennary glycans of at least 15% of all glycans attached to the FSH in the preparation. Preferably, the relative amount of at least tetraantennary glycans is at least 16%, at least 17%, at least 18% or at least 19%, more preferably at least 20% or at least 21%. The relative amount of at least tetraantennary glycans may for example be in the range of from 10% to 50%, preferably from 12% to 40%, more preferably from 15% to 35% or from 17% to 30%. The relative amount of at least triantennary glycans, in particular tri- and tetraantennary glycans, preferably is at least 25%, at least 30%, at least 35% or at least 40%, more preferably at least 45%. The relative amount of at least triantennary glycans may for example be in the range of from 20% to 70%, preferably from 30% to 65%, more preferably from 35% to 60% or from 40% to 55%.

In further embodiments, the glycosylation pattern of the recombinant FSH in the preparation may further comprise a relative amount of glycans carrying galactose of at least 90% of all glycans attached to the FSH in the preparation. The relative amount of glycans carrying galactose preferably is at least 95% or at least 97%, and most preferably is about 98%. Said relative amount of glycans carrying galactose refers to all glycan carrying a galactose residue on at least one branch or antenna of the glycan structure. Since the glycan structures of FSH commonly have more than one branch, in particular three or four branches, also the number of braches carrying or not carrying a galactose unit can be determined. Preferably, the relative amount of glycan branches carrying a galactose unit optionally modified by a sialic acid residue is at least 65%, more preferably at least 70% or at least 73% of all glycan branches of all glycans attached to the FSH in the preparation. It is preferably in the range of from about 60% to about 95%, and more preferably in the range of from about 70% to about 80%.

In certain embodiments, the glycosylation pattern of the recombinant FSH in the preparation may comprise a relative amount of glycans carrying a core fucose of at least 20% of all glycans attached to the FSH in the preparation. Preferably, the relative amount of glycans carrying core fucose is at least 25%, at least 30% or at least 35%. It may be in the range of from about 30% to about 60%, in particular in the range of from about 35% to about 50%. “Core fucose” according to the invention refers to fucose residues attached to the N-acetylgalactosamine (GlcNAc) residue at the reducing end of N-linked carbohydrate chains, i.e. the N-acetylgalactosamine residue which is directly attached to the polypeptide chain of FSH. The core fucose residue preferably is linked to the GlcNAc residue via an α1,6-linkage. A core fucose residue is opposed to an outer arm fucose residue. “Outer arm fucose” as referred to herein means a fucose residue which is attached to a branch or antenna of the N-linked carbohydrate chain. In particular, the outer arm fucose is attached to a GlcNAc residue present in the antennae, preferably via an α1,3-linkage. In specific embodiments, the glycosylation pattern of the recombinant FSH in the preparation may comprise a relative amount of glycans carrying an outer arm fucose of 5% or less of all glycans attached to the FSH in the preparation. Preferably, the relative amount of glycans carrying outer arm fucose is 4% or less, 3% or less, 2% or less or 1% or less. It may be in the range of from about 0% to about 5%, in particular in the range of from about 0% to about 2%. In certain embodiments, the recombinant FSH in the preparation does not comprise detectable amounts of outer arm fucose.

In specific embodiments, the recombinant FSH in the preparation has a diverse glycosylation pattern. The term “diverse glycosylation pattern” in particular refers to the glycosylation pattern of the FSH proteins in a preparation or composition which glycosylation pattern comprises multiple different glycan structures. Different glycan structures are oligosaccharide structures which differ in the presence/absence, amount and/or position of at least one monosaccharide unit and/or at least one chemical modification such as e.g. sulfate residues, acetyl residues or the like. A specific “different glycan structure” preferably is only considered in this respect if its relative amount is at least 0.02%, more preferably at least 0.03%, at least 0.05%, at least 0.07%, at least 0.1%, at least 0.15%, at least 0.2%, at least 0.25%, at least 0.3% or at least 0.5% of the total amount of glycan structures in the glycosylation pattern. A diverse glycosylation pattern in particular is a glycosylation pattern which comprises at least 5 different glycan structures. Preferably, the diverse glycosylation pattern comprises at least 7, more preferably at least 10, at least 15, at least 20, at least 25, at least 30, at least 35, at least 40, at least 45, at least 50, at least 55 and most preferably at least 60 different glycan structures. According to one embodiment, a diverse glycosylation pattern in particular also refers to a glycosylation pattern of FSH in a preparation or composition which glycosylation pattern comprises more different glycan structures than FSH obtained from CHO cells in a respective preparation or composition. In particular, the glycosylation pattern comprises at least 10%, preferably at least 20%, at least 30%, at least 40%, at least 50%, at least 60%, at least 70%, at least 80%, at least 90%, and most preferably at least 100% more different glycan structures than FSH obtained from CHO cells. In particular embodiments, the FSH in the preparation preferably has a diverse glycosylation pattern wherein the FSH in the preparation comprises at least 45 or preferably at least 50 different glycan structures, wherein each one of the different glycan structures has a relative amount of at least 0.05% of the total amount of glycan structures of the FSH in the preparation. According to one embodiment, the FSH in the preparation comprises at least 35 or preferably at least 40 different glycan structures, wherein each one of the different glycan structures has a relative amount of at least 0.1% of the total amount of glycan structures of the FSH in the preparation; and/or the FSH in the preparation comprises at least 20 or preferably at least 25 different glycan structures, wherein each one of the different glycan structures has a relative amount of at least 0.5% of the total amount of glycan structures of the FSH in the preparation. In a further embodiment, the FSH in the preparation comprises at least 40%, preferably at least 50% more different glycan structures than FSH obtained from CHO cells in a corresponding preparation, wherein each one of the different glycan structures has a relative amount of at least 0.05%, 0.1% or 0.5% of the total amount of glycan structures of the FSH in the respective preparation. The term “CHO” as used herein preferably refers to the CHO cell line CHOdhfr− [ATCC No. CRL-9096].

In certain embodiments, the recombinant FSH preparation according to the invention does not comprise N-glycolyl neuraminic acids (NeuGc) or detectable amounts of NeuGc. Furthermore, the recombinant FSH preparation according to the invention preferably also does not comprise Galili epitopes (Galα1,3-Gal structures) or detectable

The present invention in particular provides a FSH with a human glycosylation pattern. A human glycosylation pattern in particular is a glycosylation pattern which only comprises glycan structures which can also be found on natural human glycoproteins produced by the human body. Due to these glycosylation properties, foreign immunogenic non-human structures which may induce side effects are absent which means that unwanted side effects or disadvantages known to be caused by certain foreign sugar structures such as the immunogenic non-human sialic acids (NeuGc) or the Galili epitope (Gal-Gal structures), both known for rodent production systems, or other structures like immunogenic high-mannose structures as known from e.g. yeast systems are avoided.

In certain embodiments the glycosylation pattern of the recombinant FSH in the preparation according to the present invention comprises one or more, preferably two or more or three or more, most preferably all of the following characteristics:
(i) a relative amount of glycans carrying bisecting N-acetylglucosamine (bisGlcNAc) in the range of from about 25% to about 50% of all glycans attached to the FSH in the preparation;
(ii) a relative amount of sulfated glycans of at least 6% of all glycans attached to the FSH in the preparation;
(iii) a relative amount of 2,6-coupled sialic acid of at least 53% of all sialic acid residues attached to the FSH in the preparation;
(iv) a relative amount of glycans carrying one or more sialic acid residues of at least 88% of all glycans attached to the FSH in the preparation;
(v) a relative amount of at least tetraantennary glycans of at least 16% of all glycans attached to the FSH in the preparation; and
(vi) the FSH has a Z-number of at least 210.

In further embodiments, the recombinant FSH preparation according to the invention comprises one or more, preferably at least two, more preferably all of the following characteristics
(i) it is human recombinant FSH; and/or
(ii) it is produced by a human cell line or human cells; and/or
(iii) it has a diverse glycosylation pattern and preferably comprises at least 20 different glycan structures, wherein each one of the different glycan structures has a relative amount of at least 0.1% of the total amount of glycan structures of the FSH in the preparation.

In particular, the recombinant FSH preparation according to the invention has a glycosylation pattern which comprises one or more, preferably at least two, more preferably at least three or at least four, most preferably all of the following characteristics:
(i) a relative amount of glycans carrying bisecting N-acetylglucosamine (bisGlcNAc) in the range of from about 25% to about 50% of all glycans attached to the FSH in the preparation;
(ii) a relative amount of 2,6-coupled sialic acid in the range of from about 53% to about 80% of all sialic acid residues attached to the FSH in the preparation;
(iii) a relative amount of glycans carrying a sulfate group in the range of from about 6% to about 25% of all glycans attached to the FSH in the preparation;
(iv) a relative amount of glycans carrying outer arm fucose of 5% or less of all glycans attached to the FSH in the preparation;
(v) a relative amount of glycans carrying core fucose of at least 30% of all glycans attached to the FSH in the preparation;
(vi) a relative amount of at least tetraantennary glycans of at least 16% of all glycans attached to the FSH in the preparation;
(vii) a relative amount of glycans carrying one or more sialic acid residues of at least 88% of all glycans attached to the FSH in the preparation;
(viii) a Z-number of at least 210;
(ix) a relative amount of glycans carrying galactose of at least 95% of all glycans attached to the FSH in the preparation;
(x) a relative amount of glycan branches carrying a galactose unit optionally modified by a sialic acid residue of at least 60% of all glycan branches attached to the FSH in the preparation;
(xi) it comprises at least 45 different glycan structures, wherein each one of the different glycan structures has a relative amount of at least 0.05% of the total amount of glycan structures of the FSH in the preparation;
(xii) it comprises at least 35 different glycan structures, wherein each one of the different glycan structures has a relative amount of at least 0.1% of the total amount of glycan structures of the FSH in the preparation; and/or
(xiii) it comprises at least 20 different glycan structures, wherein each one of the different glycan structures has a relative amount of at least 0.5% of the total amount of glycan structures of the FSH in the preparation.

In certain preferred embodiments, the recombinant FSH preparation has one of the glycosylation patterns listed in the following Table 1:

TABLE 1
Specific glycosylation parameters

| Embodiment | B | 2,6-S | sulfate | S > 0 | Z | tetra |
|---|---|---|---|---|---|---|
| 1 | ≧20 | ≧53 | ≧2.5 | | | |
| 2 | ≧20 | ≧53 | ≧2.5 | ≧80 | ≧200 | ≧15 |
| 3 | ≧20 | ≧53 | ≧2.5 | ≧85 | | |
| 4 | ≧20 | ≧53 | ≧2.5 | | ≧220 | |
| 5 | ≧20 | ≧53 | ≧2.5 | | | ≧17 |
| 6 | ≧20 | ≧53 | ≧2.5 | ≧85 | ≧220 | ≧17 |
| 7 | 20-50 | ≧53 | ≧2.5 | | | |
| 8 | ≧20 | 53-80 | ≧2.5 | | | |
| 9 | ≧20 | ≧53 | 2.5-30 | | | |
| 10 | ≧20 | ≧53 | ≧2.5 | ≧80 | 200-260 | ≧15 |
| 11 | ≧20 | ≧53 | ≧2.5 | ≧80 | ≧200 | 15-30 |
| 12 | 20-50 | 53-80 | 2.5-30 | 80-100 | 200-260 | 15-30 |
| 13 | ≧25 | ≧55 | ≧3 | | | |
| 14 | ≧30 | ≧55 | ≧3 | | | |
| 15 | ≧25 | ≧55 | ≧8 | | | |
| 16 | ≧25 | ≧55 | ≧3 | ≧80 | ≧200 | ≧15 |
| 17 | ≧25 | ≧55 | ≧3 | ≧85 | ≧220 | ≧17 |

shown are the relative amounts of glycans having the following property:
B: bisecting GlcNAc;
2,6-S: 2,6-coupled sialic acid;
sulfate: sulfated glycans;
S > 0: at least one sialic acid;
Z: Z number;
tetra: at least tetraantennary glycans

In embodiments 1 to 12 listed in table 1, preferably the relative amount of bisecting GlcNAc is at least 25% instead of at least 20%; and/or the relative amount of 2,6-coupled sialic acids preferably is at least 55% instead of at least 53%; and/or the relative amount of sulfated glycans preferably is at least 3%, more preferably at least 8%, instead of at least 2.5%. The glycosylation patterns listed in table 1 preferably are human glycosylation patterns and/or do not comprise NeuGc and the Galili epitope.

In certain embodiments, the FSH is not modified by unnatural molecules, in particular by molecules which are not attached to it by the host cells used for the recombinant production. Preferably, the FSH used herein does not comprise or is not conjugated to molecules such as polyethylene glycol or hydroxyethyl starch or other molecules which are used for extending the half-life of the FSH. These molecules are in particular used in the prior art to artificially increase the circulation half-life of the FSH in the human body. However, these approaches are problematic since the polymeric substances attached to the FSH or their digestion products may cause adverse reactions in the patient, e.g. by being toxic or causing unwanted immune reactions. Furthermore, a high circulation half-life may cause the FSH to remain in the human body long after the end of the treatment. Hence, a controlled treatment is much more difficult to achieve using FSH having a high circulation half-life. In certain embodiments, the amino acid sequence of the FSH is also not artificially engineered so as to extend its circulation half-life in the human body. In particular, the FSH according to the invention is not a chimeric protein and/or does not contain glycosylation sites which are not present in the natural FSH protein.

In specific embodiments, the recombinant FSH preparation according to the invention has a circulation half-life (t₁₁₂) in humans of 50 h or less, preferably 45 h or less or even 40 h or less. Preferably, the circulation half-life of the recombinant FSH preparation according to the invention is in the range of from 20 h to 60 h, more preferably from 25 h to 50 h or from 30 h to 45 h. In further embodiments, the recombinant FSH preparation according to the invention has a lower circulation half-life than FSH preparations obtained from human urine. The circulation half-life in particular is determined in humans. Preferably, the circulation half-life is at least 5% lower, more preferably at least 10%, at least 15% or at least 20% lower than that of FSH preparations obtained from human urine. In certain embodiments, the recombinant FSH preparation according to the invention has a lower bioavailability than FSH preparations obtained from human urine and/or expressed in CHO cells, in particular, in one or more of humans, cynomolgus monkeys, rats and/or mice. Preferably, the bioavailability is at least 5% lower, more preferably at least 10%, at least 15% or at least 20% lower than that of FSH preparations obtained from human urine and/or expressed in CHO cells. Bioavailability in this respect preferably refers to the area under the curve (AUC) value obtained in pharmacokinetic studies wherein the serum FSH concentration is determined at different time points after administration of a defined amount of FSH. Circulation half-life and bioavailability preferably are determined after administration of the FSH by subcutaneous injection, in particular after single dose administration, wherein the single dose preferably comprises about 10 to about 1000 IU FSH, more preferably about 25 IU to about 500 IU FSH, about 50 IU to about 300 IU FSH or about 75 IU to about 150 IU FSH, in particular about 100 IU FSH.

In particular, circulation half-life and bioavailability are determined as disclosed in Example 4, below.

The FSH preparation obtained from human urine in particular is obtained from urine of post-menopause women. The FSH preparation expressed in CHO cells is for example expressed in the CHO cell line CHOdhfr− [ATCC No. CRL-9096]. The FSH preparation obtained from human urine and the FSH preparation expressed in CHO cells preferably are commercially available and approved pharmaceutical preparations, in particular Bravelle and Gonal-f, respectively. When comparing the circulation half-life or bioavailability of different FSH preparations, the FSH preparations are analyzed by administering them to similar subject groups with the same dosage regimen using the same administration pathway.

### Production of FSH

The FSH used according to the invention preferably is FSH, more preferably human FSH, obtainable by recombinant production in a human cell, preferably a human cell line. The human cell line that can be used as host cell for recombinant production preferably is derived from a human blood cell, in particular it is a myeloid cell line, preferably a myeloid leukemia cell line. The cell line preferably is immortalized. In a preferred embodiment, the cell line for the production of the FSH according to the invention is the cell line GT-5s, deposited on Jul. 28, 2010 under the accession number DSM ACC3078 according to the requirements of the Budapest Treaty at the Deutsche Sammlung von Mikroorganismen und Zellkulturen (DSMZ), Inhoffenstraβe 7B, 38124 Braunschweig (DE) by the Glycotope GmbH, Robert-Rössle-Str. 10, 13125 Berlin (DE), or a cell line derived therefrom, or a homologous cell line. GT-5s is an immortalized human myeloid leukemia cell line which is capable of providing the specific glycosylation pattern as described herein. According to the present invention, the terms GT5s and GT5s cell line” also include cells or cell lines derived from GT-5s. A cell line which is derived from GT-5s can be for example obtained by randomly or specifically selecting a single clone or a group of cells from a GT-5s culture, optionally after treating the GT-5s cells in order to enhance their mutation rate, or by genetically altering a GT-5s cell line. The selected clone or group of cells may further be treated as described above and/or further rounds of selection may be performed. A cell line which is homologous to GT-5s in particular is an immortalized human myeloid cell line. Preferably, a cell line derived from or homologous to GT-5s is capable of providing FSH having a glycosylation pattern similar to that obtained from GT-5s. Preferably, FSH that is produced by a cell line derived from or homologous to GT-5s has one or more of the glycosylation characteristics as described herein, in particular a relative amount of glycans carrying bisecting N-acetylglucosamine (bisGlcNAc) of at least 20% of all glycans attached to the FSH in the preparation; and/or a relative amount of glycans carrying core fucose of at least 30% of all glycans attached to the FSH in the preparation; and/or a relative amount of 2,6-coupled sialic acid of at least 40% of all sialic acid residues attached to the FSH in the preparation. According to one embodiment, the cell line derived from or homologous to GT-5s is capable of expressing FSH having a glycosylation pattern as is described as preferred herein, in particular a glycosylation pattern selected from Table 1. The similar glycosylation pattern of FSH that is produced by the cell line derived from or homologous to GT-5s is preferably similar to the glycosylation pattern of FSH obtained from GT-5s and in particular differs therefrom by not more than 20% or less, more preferably 15% or less, 10% or less or 5% or less, in particular in one or more, preferably all of the glycosylation properties selected from the group consisting of the relative amount of bisGlcNAc, the relative amount of sialylated glycans, the relative amount of sulfated glycans, the relative amount of 2,6-coupled sialic acids, the relative amount of fucose, the relative amount of tetraantennary glycans, the relative amount of glycan branches carrying galactose, and the Z number. Furthermore, the FSH according to the invention preferably is FSH, more preferably human FSH, having one or more specific glycosylation characteristics as disclosed herein, preferably a glycosylation pattern selected from Table 1. The cell line GT-5s as well as cell lines derived therefrom and cell lines homologous thereto are in particular advantageous since they provide a very stable and homogeneous protein production, in particular with respect to FSH protein. They have a very good batch-to-batch consistency, i.e. the produced proteins and their glycosylation pattern are similar when obtained from different production runs or when produced at different scales and/or with different culturing procedures. In particular, the diverse glycosylation pattern as described herein is highly reproducible in different production runs using these cell lines for expressing FSH.

It was found that an FSH produced in said cell lines exhibits a glycosylation pattern as described above and in particular exhibits the advantageous therapeutic and pharmacological activities and characteristics described herein. The recombinant FSH preparation can be produced by recombinantly expressing the FSH in a suitable cell line, in particular a cell line as described above, preferably the cell line GT-5s, a cell line derived from GT-5s or a cell line homologous to GT-5s. The recombinant FSH respectively produced can be isolated and optionally be purified.

Thus, the recombinant FSH preparation preferably is obtainable by a process comprising the steps of:
(i) cultivating a human host cell, preferably derived from the cell line GT-5s or a homologous cell line, comprising nucleic acids coding for the FSH alpha and beta subunits under conditions suitable for expression of the FSH; and
(ii) isolating FSH.

The human host cells used for expression preferably are myeloid cells, in particular immortalized myeloid leukemia cells, and preferably are or are derived from the cell line GT-5s or is a cell line homologous thereto. The human host cells are cultured so that they express FSH. Suitable culture conditions are known to the skilled person.

The term “nucleic acid” includes single-stranded and double-stranded nucleic acids and ribonucleic acids as well as deoxyribonucleic acids, in particular deoxyribonucleic acids. The term “vector” is used herein in its most general meaning and comprises any intermediary vehicle for a nucleic acid which enables said nucleic acid, for example, to be introduced into prokaryotic and/or eukaryotic host cells and, where appropriate, to be integrated into a genome of the host cell. Vectors of this kind are preferably replicated and/or expressed in the host cells. A vector preferably comprises one or more selection markers for selecting host cells comprising the vector. Suitable selection markers are resistance genes which provide the host cell with a resistance e.g. against a specific drug such as e.g. an antibiotic. Further suitable selection markers are, for example, genes for enzymes such as DHFR or GS. Vectors enabling the expression of recombinant proteins including FSH as well as suitable expression cassettes and expression elements which enable the expression of a recombinant protein with high yield in a host cell are well known in the prior art and are also commercially available, and thus, need no detailed description here. Such vectors can be used to introduce the nucleic acids encoding the amino acid sequences of FSH into host cells for recombinant expression of FSH.

The terms “cell” and “cells” and “cell line” used interchangeably, preferably refer to one or more mammalian cells, in particular human cells. The term includes progeny of a cell or cell population. Those skilled in the art will recognize that “cells” include progeny of a single cell, and the progeny can not necessarily be completely identical (in morphology or of total DNA complement) to the original parent cell due to natural, accidental, or deliberate mutation and/or change. “Cell” preferably refers to isolated cells and/or cultivated cells which are not incorporated in a living human or animal body.

The isolation of FSH preferably comprises the further steps of:
(a) obtaining the culture supernatant where the FSH is secreted by the human cells, or lysing the human cells where the FSH is not secreted;
(b) isolating the FSH from the culture supernatant or cell lysate using chromatographic steps such as reversed phase chromatography, size exclusion chromatography and/or hydrophobic interaction chromatography;
(c) optionally obtaining an acidic fraction of the FSH by removing basic FSH isoforms, preferably by using anion exchange chromatography including a washing step which removes basic FSH isoforms, such as a washing step at about pH 5.0 or about pH 4.5 or about pH 4.0.

Preferably, the nucleic acid coding for the FSH alpha subunit and the nucleic acid coding for the FSH beta subunit are comprised in expression cassettes comprised in a suitable expression vector that allows the expression in a human host cell. The nucleic acid coding for the FSH alpha subunit and the nucleic acid coding for the FSH beta subunit may be comprised in the same vector, but preferably are comprised in separate vectors which can be introduced into the host cells by co-transfection. Furthermore, they may also be expressed from one expression cassette using appropriate elements such as an IRES element. Preferably, the FSH is secreted by the human cells. In preferred embodiments, cultivation of the human cells is performed in a fermenter and/or under serum-free conditions.

A suitable purification process for the recombinant FSH is described, for example, in the PCT patent application no. WO 2011/063943.

In certain embodiments of the present invention, the recombinant FSH is recombinant human FSH (rhFSH), preferably obtainable by production in a human cell line, such as the cell line GT-5s, which comprises one or more nucleic acids encoding the human FSH subunits and elements for expressing said one or more nucleic acids in the host cell. Preferably, the alpha subunit of the rhFSH has the amino acid sequence according to SEQ ID NO: 1 or an amino acid sequence having a homology or preferably identity to SEQ ID NO: 1 over its entire length of at least 80%, preferably at least 85%, at least 90%, at least 95% or at least 98%. In preferred embodiments, the alpha subunit of the rhFSH comprises asparagine residues at positions corresponding to positions 52 and 78 of SEQ ID NO: 1 and is glycosylated at the asparagine residues corresponding to Asn52 and Asn78 of SEQ ID NO: 1. The alpha subunit of the rhFSH preferably only comprises these two glycosylation sites and does not comprise any further glycosylation sites. The beta subunit of the rhFSH preferably has the amino acid sequence according to SEQ ID NO: 2 or an amino acid sequence having a homology or preferably identity to SEQ ID NO: 2 over its entire length of at least 80%, preferably at least 85%, at least 90%, at least 95% or at least 98%. In preferred embodiments, the beta subunit of the rhFSH comprises asparagine residues at positions corresponding to positions 7 and 24 of SEQ ID NO: 2 and is glycosylated at the asparagine residues corresponding to Asn7 and Asn24 of SEQ ID NO: 2. The beta subunit of the rhFSH preferably only comprises these two glycosylation sites and does not comprise any further glycosylation sites. In certain embodiments, the FSH consists of one alpha subunit and one beta subunit and does not comprise any further amino acid sequences.

The FSH Composition

The recombinant FSH preparation preferably is present in a pharmaceutical composition. The term “pharmaceutical composition” particularly refers to a composition suitable for administering to a human or animal, i.e., a composition containing components which are pharmaceutically acceptable. Preferably, the pharmaceutical composition comprises the FSH as an active compound or a salt or prodrug thereof together with a carrier, diluent or pharmaceutical excipient such as buffer, preservative and tonicity modifier. The pharmaceutical composition preferably is a composition suitable for injection, such as subcutaneous injection or intravenous injection, for example an aqueous solution comprising the FSH, or a composition which can be used to prepare a composition suitable for intravenous injection, for example a lyophilized FSH composition. The pharmaceutical composition may include further pharmaceutically active agents, in particular further agents useful in infertility treatment such as gonadotropin-releasing hormon (GnRH) agonists or gonadotropin-releasing hormon (GnRH) antagonists. Exemplary GnRH agonists are the natural GnRH decapeptide or modified peptides such as leuprolide, buserelin, histrelin, goserelin, deslorelin, nafarelin and triptorelin. Exemplary GnRH antagonists include cetrorelix, ganirelix, abarelix and degarelix. Alternatively, the pharmaceutical composition comprising the recombinant FSH may be designed for use in combination with such further pharmaceutically active agents. In preferred embodiments, the FSH preparation does not comprise any further pharmaceutically active agents or any other gonadotropins such as LH and CG.

The pharmaceutical composition may be in the form of a single unit dose or a multiple unit dose. Preferably, the pharmaceutical composition is a sterile solution comprising the recombinant FSH according to the present invention, further comprising one or more ingredients selected from the group consisting of solvents such as water, buffer substances, stabilizers, preservatives, excipients, surfactants and salts. A multiple unit dose comprises enough FSH to provide for multiple single doses, in particular at least 3, at least 4, at least 5, at least 6, at least 7, at least 8, at least 9, at least 10, at least 15, at least 20 or at least 50 single doses. The pharmaceutical composition may for example be in the form of an injection pen. The components of the composition preferably are all pharmaceutically acceptable. The composition may be a solid or fluid composition, in particular a—preferably aqueous—solution, emulsion or suspension or a lyophilized powder.

The pharmaceutical composition preferably comprises the FSH in a concentration in the range of from 1 to 5000 IU/ml, more preferably from 10 to 2500 IU/ml, from 100 to 2000 IU/ml or from 250 to 1500 IU/ml, in particular about 500 IU/ml or about 1000 IU/ml.

Preferably, the recombinant FSH preparation according to the present invention is for parenteral administration to the patient. In particular, the recombinant FSH is to be administered by injection or infusion, for example intravenously, intramuscularly or subcutaneously.

The Method for Controlled Ovarian Hyperstimulation

The present invention is directed to methods for controlled ovarian hyperstimulation. Said controlled ovarian hyperstimulation includes stimulation of the development of multiple ovarian follicles in a female subject. Said development of multiple ovarian follicles is achieved simultaneously in one single cycle. Controlled ovarian hyperstimulation in particular refers to the stimulation of the development of a higher number of follicles than would occur naturally. The development of ovarian follicles especially leads to the formation of cumulus oocyte complexes (COCs) and metaphase II oocytes. The cumulus oocyte complex is a cluster of cells comprising the oocyte and cumulus cells surrounding it. It is part of the ovarian follicle prior to ovulation and during ovulation leaves the ruptures follicle to enter the fallopian tube. Metaphase II oocytes are arrested in the metaphase of the second meiosis of the oocyte development.

In specific embodiments, the controlled ovarian hyperstimulation is part of an assisted reproductive technology (ART). In particular, it may be combined with in vitro fertilization such as intracytoplasmatic sperm injection, co-incubation of oocyte and sperms and gamete intrafallopian transfer; and/or embryo transfer such as zygote intrafallopian transfer.

The method for controlled ovarian hyperstimulation for stimulating the development of multiple ovarian follicles in a female subject in particular comprises the steps of
(a) administering to a female subject a recombinant FSH preparation;
(b) triggering ovulation; and
(c) obtaining multiple oocytes from the female subject.

The Dosage Regimen

In step (a), a recombinant FSH preparation is administered to a female subject using a specific dosage regimen. In a first aspect, the dosage regimen includes the administration of the recombinant FSH preparation so that the single doses sum up to an average amount of from about 35 to about 250 IU FSH per day. In a second aspect of the present invention, the dosage regimen includes the administration of the recombinant FSH preparation in an amount in IU which is 80% or less of the amount recommended for recombinant FSH preparations produced by CHO cells in the same therapeutic situation.

Different dosage intervals may be used. In particular, the recombinant FSH preparation may be administered several times a day, such as four times a day, three times a day or twice daily, once each day, or every second day, every third day, every fourth day or every fifth day. Preferably, the recombinant FSH preparation is administered once each day, every second day or every third day, in particular once each day or every second day, especially once each day. The sum of the amount of FSH in each single dose divided by the days of the administration results in the average amount of FSH per day. Hence, an average amount of 100 IU FSH per day can be reached, for example, by administering single doses of (a) 50 IU FSH twice daily, (b) 100 IU FSH daily, (c) 200 IU FSH every second day, or (d) 300 IU FSH every third day.

In specific embodiments, a dosage regimen is used wherein the single doses sum up to an average amount of from about 35 to about 150 IU FSH per day. In particular, the average amount is from about 45 to about 125 IU FSH per day, especially from about 50 to about 115 IU FSH per day, from about 55 to about 100 IU FSH per day, or from about 60 to about 90 IU FSH per day. In certain embodiments, the dosage regimen includes daily doses of about 35 to about 150 IU FSH, preferably about 45 to about 125 IU FSH, about 50 to about 115 IU FSH, about 55 to about 100 IU FSH, or about 60 to about 90 IU FSH. In further embodiments, the dosage regimen includes doses of about 70 to about 300 IU FSH every second day, preferably about 90 to about 250 IU FSH every second day, about 100 to about 230 IU FSH every second day, about 110 to about 200 IU FSH every second day, or about 120 to about 180 IU FSH every second day. In further embodiments, the dosage regimen includes doses of about 105 to about 450 IU FSH every third day, preferably about 135 to about 375 IU FSH every third day, about 150 to about 345 IU FSH every third day, about 165 to about 300 IU FSH every third day, or about 180 to about 270 IU FSH every third day.

In further embodiments, a dosage regimen is used wherein the single doses sum up to an average amount of from about 70 to about 300 IU FSH per day. In particular, the average amount is from about 90 to about 250 IU FSH per day, especially from about 110 to about 230 IU FSH per day, from about 125 to about 190 IU FSH per day, or from about 140 to about 160 IU FSH per day. In certain embodiments, the dosage regimen includes doses of about 70 to about 300 IU FSH every day, preferably about 90 to about 250 IU FSH every day, about 110 to about 230 IU FSH every day, about 125 to about 190 IU FSH every day, or about 140 to about 160 IU FSH every day. In further embodiments, the dosage regimen includes doses of about 140 to about 600 IU FSH every second day, preferably about 180 to about 500 IU FSH every second day, about 220 to about 460 IU FSH every second day, about 250 to about 380 IU FSH every second day, or about 280 to about 320 IU FSH every second day. In these embodiments, the female subject may in particular have a low responsiveness to stimulation of follicle growth or show a poor ovarian response to ovarian stimulation. Particularly, the female subject may be selected from the group consisting of
female subjects having an age of at least 35 years, in particular at least 37 years or at least 40 years, preferably in the range of about 38 years to about 50 years;
female subjects having a serum level of anti-mullerian hormone (AMH) of 1.5 ng/ml or less, in particular 1.4 ng/ml or less, 1.3 ng/ml or less, 1.2 ng/ml or less or 1.1 ng/ml or less, preferably in the range of about 0.25 ng/ml to about 1.25 ng/ml;
female subjects having an antral follicle count of 9 or less as the sum of both ovaries, in particular 8 or less, 7 or less or 6 or less, preferably in the range of 4 to 7; and
female subjects having a body mass index (BMI) of at least 25 kg/m², in particular at least 26 kg/m², at least 27 kg/m², at least 28 kg/m², at least 29 kg/m² or at least 30 kg/m², preferably in the range of about 28 kg/m² to about 45 kg/m²;
female subjects having undergone a previous conventional FSH stimulation cycle wherein the development of less than 6, in particular less than 5, less than 4 or less than 3 oocytes was induced; and
female subjects having a poor ovarian response according to the 2011 ESHR Bologna criteria as defined in Ferraretti et al. (2011) Human Reproduction 26(7), 1616-1624.

In specific embodiments, the female subject which has a low responsiveness to stimulation of follicle growth or shows a poor ovarian response to ovarian stimulation fulfills two or more, in particular three or more of these criteria.

In certain embodiments, the recombinant FSH preparation as described herein is administered in an amount in IU which is 75% or less, in particular 60% or less or 50% or less of the amount recommended for recombinant FSH preparations produced by CHO cells in the same therapeutic situation. The recombinant FSH preparation produced by CHO cells in particular is Gonal-f. The amount recommended for recombinant FSH preparations produced by CHO cells may be the dosage indicated in the prescription information of the recombinant FSH preparation produced by CHO cells. In other embodiments, the amount recommended for recombinant FSH particular a physician, as suitable for controlled ovarian hyperstimulation in the female subject, especially in order to stimulate the development of multiple oocytes, in particular at least 5 oocytes, in the female subject.

Preferably, an administration of the FSH preparation refers to the transfer of one dose of the FSH preparation into the body of the subject. In particular, a single dose of the FSH preparation is administered. Said dose of the FSH preparation preferably is given as a single dose, e.g. by one injection. Administration every day in particular means that at least 12 hours, preferably at least 18 hours, in particular about 24 h are between the end of one administration and the beginning of the next administration. In particular, there is no entire calendar day between the end of one administration and the beginning of the next administration. Administration every second day in particular means that at least 30 hours, preferably at least 36 hours are between the end of one administration and the beginning of the next administration. In particular, an entire calendar day is between the end of one administration and the beginning of the next administration. In particular, when administering every second day a subsequent dose of FSH is given about 42 to about 54 h, preferably about 44 h to about 52 h, more preferably about 46 h to about 50 h after the preceding dose. Administration every third day in particular means that at least 54 hours, preferably at least 60 hours are between the end of one administration and the beginning of the next administration. In particular, two entire calendar days are between the end of one administration and the beginning of the next administration. In particular, when administering every third day a subsequent dose of FSH is given about 66 to about 78 h, preferably about 68 h to about 76 h, more preferably about 70 h to about 74 h after the preceding dose. Administration every fourth day in particular means that at least 78 hours, preferably at least 84 hours are between the end of one administration and the beginning of the next administration. In particular, three entire calendar days are between the end of one administration and the beginning of the next administration. In particular, when administering every fourth day a subsequent dose of FSH is given about 90 to about 102 h, preferably about 92 h to about 100 h, more preferably about 94 h to about 98 h after the preceding dose. Administration every fifth day in particular means that at least 102 hours, preferably at least 108 hours are between the end of one administration and the beginning of the next administration. In particular, four entire calendar days are between the end of one administration and the beginning of the next administration. In particular, when administering every fifth day a subsequent dose of FSH is given about 114 to about 126 h, preferably about 116 h to about 124 h, more preferably about 118 h to about 122 h after the preceding dose.

Preferably, the FSH preparation according to the invention is administered for a time interval of at least 5 days, preferably at least 6 days, at least 7 days, at least 8 days or at least 9 days. In particular, the FSH preparation is administered for a time interval of between 5 to 21 days, preferably between 6 to 18 days.

In certain preferred embodiments, the FSH preparation according to the invention is initially administered for a time interval of about 4 to 9 days, preferably 5 to 7 days, (initial administration regimen) and thereafter the treated subject is examined for her response to the treatment. Such examination in particular includes the determination of the number and/or size of the induced follicles in one or both ovaries. The further treatment may then be adjusted on the basis of the results of the examination, for example to continue the follicle growth stimulation or even to increase the follicle growth stimulation. For example, the FSH treatment may be stopped if enough large follicles for the intended purpose are detected, or one or more, preferably two, three, four, five, six or more doses of FSH may be administered subsequently. The subsequent administration regimen may be the same as or different from the administration regimen prior to the examination. The further FSH doses may be the same as or may be different from the FSH doses that were administered prior to the examination. For example, the administered doses may contain an amount of FSH in the range of from about 50% to about 300%, preferably from about 75% to about 200%, more preferably from about 100% to about 150% of the dosage amount that was given prior to the examination. In preferred embodiments, the administration regimen and the dose of FSH are the same prior to and after the examination or the administration regimen is the same and the dose of FSH is increased by 50% after the examination. According to one embodiment, FSH is not administered for more than 20 days, preferably no longer than 18 days.

Preferably, the FSH is administered in a dosage regimen using single doses in the range of from about 10 to about 2000 IU FSH. The single dose used for each administration preferably comprises about 20 to about 1500 IU FSH, more preferably about 25 to about 1000 IU FSH, about 30 to about 750 IU FSH, about 37.5 to about 500 IU FSH, about 50 to about 300 IU FSH, or about 60 to about 200 IU FSH, most preferably about 75 to about 150 IU FSH. In preferred embodiments, each dose of the administration regimen or at least of the initial administration regimen contains the same amount of FSH or the amount of FSH per dose varies by no more than 10%, preferably no more than 5%.

The international units (IU) for FSH refer to the fourth International Standard for Human Urinary FSH and LH (Storring, P. L. & Gaines Das, R. E. (2001) Journal of Endocrinology 171, 119-129) and are determined according to the augmented ovarian weight gain method (Steelman, S. L. & Pohley, F. M. (1953) Endocrinology 53, 604-616).

The method of controlled ovarian hyperstimulation may further include down-regulation of the natural menstruation cycle prior to and optionally also during the administration of the recombinant FSH preparation. The down-regulation of the natural menstruation cycle can be achieved by treatment of the female subject with either a gonadotropin-releasing hormone agonist (GnRH-agonist) or a gonadotropin-releasing hormone antagonist (GnRH-antagonist), which both ultimately result in a decreased serum level of natural luteinising hormone (LH) and natural follicle-stimulating hormone (FSH). GnRH-agonists strongly bind to and activate the gonadotropin-releasing hormone receptor and hence, cause constant stimulation of the pituitary. As a result, initially there is an increase in FSH and LH secretion (so-called “flare effect”). However after about ten days a profound hypogonadal effect (i.e. decrease in FSH and LH) is achieved through receptor downregulation by internalization of receptors. Suitable GnRH-agonists are, for example, triptorelin, leuprolide, buserelin, nafarelin, histrelin, goserelin and deslorelin. A GnRH-agonist may be given e.g. starting at day 20 or 22 of the menstruation cycle. GnRH-antagonists competitively bind to GnRH receptors in the pituitary gland, thereby blocking their activation and hence, the release of natural luteinising hormone (LH) and natural follicle-stimulating hormone (FSH) from the pituitary. Suitable GnRH-antagonists are, for example, cetrorelix, ganirelix, abarelix and degarelix. Administration of the recombinant FSH preparation preferably begins after down-regulation of the FSH and LH level is achieved, usually after about 8 to 25 days from the beginning of the down-regulation treatment. The treatment with the GnRH-agonist or GnRH-antagonist may be continued during the FSH treatment. Respective treatments are well known in the prior art and thus, do not need a detailed description.

In certain embodiments, the administration of the recombinant FSH preparation in step (a) does not comprise the concurrent administration of another gonadotropin such as LH or CG or another agent which induces or enhances follicle growth.

The term “the same therapeutic situation” as used herein refers to a situation wherein a similar female subject is treated similar to the reference situation. In particular, in the compared situations the female subject is similar with respect to the conditions relevant for fertility treatment such as the age, the serum level of anti-mullerian hormone, the antral follicle count, the body mass index and previous infertility treatments such as previous conventional FSH stimulation cycles. Furthermore, in the compared situations preferably the treatment is similar, including the dose schedule, the method used for obtaining the oocytes, the subsequent treatment of the retrieved oocytes, the pretreatment of the female subject, any concomitant treatments with other agents, etc., except indicated otherwise. Similar in this respect in particular refers to deviations in numbers of 25% or less, preferably 10% or less, in particular 5% or less. In case of features which cannot be expressed in numbers, the situation preferably is the same. Generally, the skilled practitioner is able determine whether two therapeutic situations are the same.

Induction of Ovulation

In step (b) of the method according to the present invention, ovulation is triggered in the female subject. In particular, ovulation is triggered when there are multiple follicles with a mean diameter equal to or greater than 12 mm and/or when there is at least one follicle with a diameter of at least 17 mm. In certain embodiments, ovulation is triggered when there are multiple follicles, in particular at least 3, preferably at least 4, at least 5 or at least 6 follicles, with a mean diameter equal to or greater than 12 mm, in particular with a mean diameter equal to or greater than 13 mm or 14 mm. In further embodiments, ovulation is triggered when there is at least one follicle with a diameter of at least 17 mm, in particular with a diameter of at least 18 mm, at least 19 mm or at least 20 mm. In specific embodiments, ovulation is triggered when there are multiple follicles, in particular at least 3, preferably at least 4, at least 5 or at least 6 follicles, with a mean diameter equal to or greater than 12 mm, in particular with a mean diameter equal to or greater than 13 mm or 14 mm, and when there is at least one follicle with a diameter of at least 17 mm, in particular with a diameter of at least 18 mm, at least 19 mm or at least 20 mm. The number and size of the follicles may be determined by means of ultrasound analysis such as gynecologic ultrasonography.

Triggering ovulation in particular is achieved by administration of an ovulation inducer to the female subject. Suitable ovulation inducers are chorionic gonadotropin, in particular human chorionic gonadotropin (hCG) such as recombinant hCG, luteinizing hormone (LH) such as recombinant LH, GnRH agonists, or derivatives thereof. The ovulation inducer is preferably administered after the treatment with FSH is stopped.

In particular, the ovulation inducer may be administered 6 to 72 hours, preferably 12 to 54 hours, especially 18 to 36 hours after the last FSH administration. In certain specific embodiments, administration of the ovulation inducer is commenced at least 48 h after termination of the administration of the recombinant FSH preparation, especially about 60 h to about 120 h, or about 72 h to about 96 h after termination of the administration of the recombinant FSH preparation. Preferably, about 100 to 500 μg, more preferably 200 to 300 μg, in particular about 250 μg hCG or its derivative is administered. Triggering ovulation in this respect in particular includes the induction of meiosis II of an oocyte, the stimulation of the development of an oocyte in the metaphase II stage, and/or the induction of ovulation itself.

The step of triggering ovulation does not have to include the actual ovulation of one or more follicles. It in particular refers to the induction of the ovulation process, including for example the final maturation of oocytes. Obtaining the oocytes in step (c) in specific embodiments is performed prior to the completion of the ovulation process.

Retrieval of the Oocytes

In step (c) of the method for controlled ovarian hyperstimulation, multiple oocytes are obtained from the female subject. In a first aspect of the present invention, on average at least 5 oocytes per female subject are obtained and/or at least 5 oocytes from the female subject are obtained. In a second aspect of the present invention, on average at least 5% more oocytes per female subject are obtained compared to a similar treatment with the amount recommended for recombinant FSH preparations produced by CHO cells in the same therapeutic situation.

In particular embodiments, on average at least 5 oocytes are obtained per female subject. Especially, on average at least 6, preferably at least 7, at least 8, at least 9 or at least 10 oocytes are obtained per female subject. The average number of oocytes per female subject is determined by dividing the sum of all oocytes obtained from a group of female subjects by the number of female subjects. All female subjects were treated with the same dosage regimen using the recombinant FSH preparation as described herein. The group of female subjects encompasses at least 20 subjects, preferably at least 40 subjects or at least 100 subjects. In further embodiments, at least 5 oocytes are obtained from the female subject to whom the recombinant FSH preparation was administered in step (a). Especially, at least 6, preferably at least 7, at least 8, at least 9 or at least 10 oocytes are obtained from the female subject.

In further embodiments, on average at least 5% more, in particular at least 6% more, at least 7% more, at least 8% more, at least 10% more or at least 15% more oocytes per female subject are obtained compared to a similar treatment with the amount recommended for recombinant FSH preparations produced by CHO cells in the same therapeutic situation. The recombinant FSH preparation produced by CHO cells in particular is Gonal-f. The amount recommended for recombinant FSH preparations produced by CHO cells may be the dosage indicated in the prescription information of the recombinant FSH preparation produced by CHO cells. In other embodiments, the amount recommended for recombinant FSH preparations produced by CHO cells is the amount determined by a skilled person, in particular a physician, as suitable for controlled ovarian hyperstimulation in the female subject, especially in order to stimulate the development of multiple oocytes, in particular at least 5 oocytes, in the female subject. In certain embodiments, on average at least 5% more, in particular at least 6% more, at least 7% more, at least 8% more, at least 10% more or at least 15% more cumulus oocyte complexes and/or on average at least 5% more, in particular at least 6% more, at least 7% more, at least 8% more, at least 10% more or at least 15% more metaphase II oocytes per female subject are obtained compared to a similar treatment with the amount recommended for recombinant FSH preparations produced by CHO cells in the same therapeutic situation.

The oocytes may be obtained from the female subject by surgery, in particular by puncture such as ultrasound-guided puncture. A suitable method for obtaining the oocytes is transvaginal ovum retrieval. In particular, the obtained oocytes have a mean diameter of at least 10 mm, preferably at least 12 mm. In certain embodiments, the oocytes are obtained in the form of cumulus oocyte complexes (COCs). In specific embodiments, at least part of the obtained oocytes are metaphase II oocytes, i.e. oocytes arrested in the metaphase of the second meiosis. In particular, at least 2, preferably at least 3, at least 4 or at least 5 of the obtained oocytes are metaphase II oocytes.

The oocytes preferably are obtained about 24 h to about 38 h, in particular about 32 h to about 36 h after triggering ovulation. In certain embodiments, the oocytes are obtained from the follicles after ovulation is triggered in step (b), but prior to the completion of the ovulation process, in particular prior to rupture of the follicle.

Further Method Steps

The method for controlled ovarian hyperstimulation in certain embodiments may comprise the following further method steps:
(d) fertilizing at least one oocyte obtained in step (c); and
(e) transferring at least one fertilized oocyte obtained in step (d) or at least one embryo derived therefrom into a female patient.

Fertilization of the at least one oocyte is in particular achieved by in vitro fertilization with intracytoplasmatic sperm injection (IVF-ICSI) or co-incubation with sperms (IVF). Co-incubation of oocyte and sperm can take place as well in the fallopian tube which is called gamete intrafallopian transfer. Fertilization may be monitored by detecting the presence of two pronuclei (2PN) oocytes. In specific embodiments, one, two or three oocytes are fertilized. In other embodiments, at least 4 or at least 5, in particular all oocytes obtained in step (c) are fertilized.

In step (e), in particular one, two or three fertilized oocytes or embryos derived therefrom are transferred into the female patient. In certain embodiments, only a part of the oocytes fertilized in step (d) are transferred. The female subject from whom the oocytes are obtained may be the same as or different from the female patient into whom the fertilized oocytes or embryos derived therefrom are transferred.

In certain embodiments, the method for controlled ovarian hyperstimulation further comprises freezing or vitrificating at least one oocyte obtained in step (c), in particular prior to step (d). In specific embodiments, all oocytes obtained in step (c) are frozen or vitrificated. In other embodiments, only a subset of the oocytes obtained in step (c), in particular those which are not fertilized in step (d) or only those which are fertilized in step (d), are frozen or vitrificated. Freezing or vitrificating the obtained oocytes can be used for safe storage of the oocytes and/or for enhancing the efficacy of implantation of the embryo and/or increasing the pregnancy rate. Alternatively or additionally, the method may further comprise freezing or vitrificating at least one fertilized oocyte obtained in step (d) or at least one embryo derived therefrom, in particular prior to step (e). In specific embodiments, all fertilized oocytes/embryos obtained in step (d) are frozen or vitrificated. In other embodiments, only a subset of the fertilized oocytes/embryos obtained in step (d), in particular those which are not transferred into the female patient in step (e) or only those which are transferred into the female patient in step (e), are frozen or vitrificated. In particular embodiments, only a subset of the oocytes obtained in step (c) are fertilized in step (d), and/or only a subset of the oocytes fertilized in step (d) are transferred into a female patient in step (e). Optionally, the oocytes not fertilized in step (d) or the fertilized oocytes or embryos not transferred into a female patient in step (e) are frozen or vitrificated for subsequent use.

In specific embodiments, the method for controlled ovarian hyperstimulation comprises one or more further steps of transferring at least one fertilized oocyte or at least one embryo derived therefrom into a female patient in addition to step (e). The fertilized oocytes may be obtained in step (d) or in one or more further steps of fertilizing at least one oocyte obtained in step (c). The female patient of the different transfer steps may be the same or different patients. In embodiments where the female patient is the same, a subsequent transfer step is performed only after completion of the previous treatment cycle, in particular after successful pregnancy, miscarriage or failure of the previous transfer. In particular, the oocytes used for these fertilization and transfer steps are all obtained in step (c) and the method does not comprise a second cycle of controlled ovarian hyperstimulation. Hence, in certain embodiments the method comprises only one cycle of controlled ovarian hyperstimulation.

In certain embodiments, the method for controlled ovarian hyperstimulation does not comprise in vitro maturation of the obtained oocytes. In particular, the obtained oocytes are not treated with agents such as hormones outside of the body of the female subject in order to stimulate further oocyte maturation.

In further embodiments, the method for controlled ovarian hyperstimulation does not comprise the administration of another gonadotropin such as LH or hCG or another agent which induces or enhances follicle growth prior to or concurrent with the administration of the recombinant FSH preparation in step (a). In certain embodiments, the recombinant FSH preparation as described herein is for use without any adjuvant stimulation, in particular without the use of clomiphene citrate. According to one embodiment, no oral ovulation induction agent is used in combination with the recombinant FSH preparation as described herein to stimulate follicle growth. According to one embodiment, the recombinant FSH preparation is used in a single agent therapy for the stimulation of the follicle growth. In particular, no ovulation induction agent is given during the recombinant FSH administration to support the follicle growth. However, after the last FSH administration an agent inducing final follicle maturation and/or triggering ovulation such as hCG may be given to the subject, in particular in step (b) of the method.

The Female Subject

In certain embodiments, the female subject is a patient suffering from a dysfunction or disease related to reproduction or fertility. The terms “subject” or “patient” according to the invention refer to a human being, a nonhuman primate or another animal, in particular a mammal such as a cow, horse, pig, sheep, goat, dog, cat or a rodent such as a mouse and rat. In a particularly preferred embodiment, the subject or patient is a human being. In case of a human subject or patient, the FSH preferably is human FSH. In specific embodiments, the female subject or female patient undergoes an assisted reproductive technology (ART). In particular, the assisted reproductive technology includes in vitro fertilization such as intracytoplasmatic sperm injection, co-incubation of oocyte and sperms and gamete intrafallopian transfer; and/or embryo transfer such as zygote intrafallopian transfer. In certain embodiments, the female subject which is subjected to the method for controlled ovarian hyperstimulation is different from the female patient into whom the fertilized oocyte or embryo is transferred. These embodiments are in particular used in egg donor programs. In other embodiments, the female subject and the female patient are the same.

In certain embodiments, the female subject has a low responsiveness to stimulation of follicle growth or shows a poor ovarian response to ovarian stimulation. Particularly, the female subject may be selected from the group consisting of
female subjects having an age of at least 35 years, in particular at least 37 years or at least 40 years, preferably in the range of about 38 years to about 50 years;
female subjects having a serum level of anti-mullerian hormone (AMH) of 1.5 ng/ml or less, in particular 1.4 ng/ml or less, 1.3 ng/ml or less, 1.2 ng/ml or less or 1.1 ng/ml or less, perferably in the range of about 0.25 ng/ml to about 1.25 ng/ml;
female subjects having an antral follicle count of 9 or less as the sum of both ovaries, in particular 8 or less, 7 or less or 6 or less, preferably in the range of 4 to 7;
female subjects having a body mass index (BMI) of at least 25 kg/m², in particular at least 26 kg/m², at least 27 kg/m², at least 28 kg/m², at least 29 kg/m² or at least 30 kg/m², preferably in the range of about 28 kg/m² to about 45 kg/m²;
female subjects having undergone a previous conventional FSH stimulation cycle wherein the development of less than 6, in particular less than 5, less than 4 or less than 3 oocytes was induced; and
female subjects having a poor ovarian response according to the 2011 ESHR Bologna criteria as defined in Ferraretti et al. (2011) Human Reproduction 26(7), 1616-1624.

In specific embodiments, the female subject which has a low responsiveness to stimulation of follicle growth or shows a poor ovarian response to ovarian stimulation fulfills two or more, in particular three or more of these criteria.

The Method for Stimulating Follicle Maturation

In a third aspect, the present invention pertains to a method for stimulating follicle maturation of in a female subject, comprising
(a) inducing or enhancing follicle growth in a female subject by administering a recombinant FSH preparation; and
(b) subsequently triggering ovulation;

wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation; and

wherein triggering ovulation in step (b) is commenced at least 48 h after termination of the administration of the recombinant FSH preparation in step (a).

In particular embodiments, ovulation is triggered when there are multiple follicles with a mean diameter equal to or greater than 12 mm and/or when there is at least one follicle with a diameter of at least 17 mm. In certain embodiments, ovulation is triggered when there are multiple follicles, in particular at least 3, preferably at least 4, at least 5 or at least 6 follicles, with a mean diameter equal to or greater than 12 mm, in particular with a mean diameter equal to or greater than 13 mm or 14 mm. In further embodiments, ovulation is triggered when there is at least one follicle with a diameter of at least 17 mm, in particular with a diameter of at least 18 mm, at least 19 mm or at least 20 mm. In specific embodiments, ovulation is triggered when there are at least 3, preferably at least 4, at least 5 or at least 6 follicles with a mean diameter equal to or greater than 12 mm, in particular with a mean diameter equal to or greater than 13 mm or 14 mm, and when there is at least one follicle with a diameter of at least 17 mm, in particular with a diameter of at least 18 mm, at least 19 mm or at least 20 mm. The number and size of the follicles may be determined by means of ultrasound analysis such as gynecologic ultrasonography. In certain embodiments, the number and size of the follicles which is decisive for triggering ovulation is determined prior to, during or at most 24 h after the last administration of the recombinant FSH preparation.

In certain embodiments, triggering ovulation in step (b) is commenced at least 54 h, in particular at least 60 h, at least 72 h, at least 84 h or at least 96 h after termination of the administration of the recombinant FSH preparation in step (a). For example, triggering ovulation in step (b) is commenced about 60 h to about 120 h, preferably about 72 h to about 96 h after termination of the administration of the recombinant FSH preparation in step (a).

Triggering ovulation in particular is achieved by administration of an ovulation inducer to the female subject. Suitable ovulation inducers are chorionic gonadotropin, in particular human chorionic gonadotropin (hCG) such as recombinant hCG, luteinizing hormone (LH), such as recombinant LH, GnRH agonists, or derivatives thereof. The ovulation inducer is preferably hGC or a derivative thereof. Preferably, about 100 to 500 μg, more preferably 200 to 300 μg, in particular about 250 μg hCG or its derivative is administered. Triggering ovulation in this respect in particular includes the induction of meiosis II of an oocyte, the stimulation of the development of an oocyte in the metaphase II stage, and/or the induction of ovulation itself.

All embodiments and features described herein with respect to the methods for controlled ovarian hyperstimulation may also likewise apply to the method for stimulating follicle maturation. Furthermore, the methods for controlled ovarian hyperstimulation may be combined with the method for stimulating follicle maturation.

### Specific Embodiments

In certain embodiments of the method for controlled ovarian hyperstimulation for stimulating the development of multiple ovarian follicles in a female subject, in step (a) a dosage regimen is used, wherein the single doses sum up to an average amount of from about 50 to about 125 IU FSH per day; in step (b) ovulation is triggered when there is at least one follicle with a diameter of at least 17 mm; and in step (c) at least 5 oocytes are obtained from the female subject in the form of cumulus oocyte complexes (COCs), and at least 4 of these oocytes are metaphase II oocytes; and the recombinant FSH in the preparation has a glycosylation pattern comprising the following characteristics:
(i) a relative amount of glycans carrying bisecting N-acetylglucosamine (bisGlcNAc) in the range of from about 25% to about 50% of all glycans attached to the FSH in the preparation;
(ii) a relative amount of 2,6-coupled sialic acid in the range of from about 53% to about 80% of all sialic acid residues attached to the FSH in the preparation;
(iii) a relative amount of sulfated glycans of at least 5% of all glycans attached to the FSH in the preparation;
(iv) a relative amount of glycans carrying outer arm fucose of 5% or less of all glycans attached to the FSH in the preparation;
(v) a relative amount of glycans carrying core fucose of at least 30% of all glycans attached to the FSH in the preparation;
(vi) a relative amount of at least tetraantennary glycans of at least 16% of all glycans attached to the FSH in the preparation;
(vii) a relative amount of glycans carrying one or more sialic acid residues of at least 88% of all glycans attached to the FSH in the preparation; and
(viii) a Z number of at least 210.

In certain embodiments, the recombinant FSH preparation described herein achieves at least the same therapeutic effects in the same therapeutic situation as Gonal f® when administered at half the dose in IU as Gonal f®. The therapeutic effect in particular includes one or more of the number of oocytes with a mean diameter equal to or greater than 12 mm induced in the female subject, the number of oocytes, COCs and/or metaphase II oocytes retrieved from the female subject, and the number of successfully fertilized embryos. The therapeutic effects are preferably determined as average in a group of female subjects, preferably comprising at least 20 female subjects, more preferably at least 40 female subjects or at least 100 female subjects. The groups are in particular comparable according to scientific standards.

The expression “comprise”, as used herein, besides it's regular meaning also includes and specifically refers to the expressions “consist essentially of” and “consist of”. Thus, according to one embodiment the expression “comprise” refers to embodiments wherein the subject-matter which “comprises” specifically listed elements does not comprise further elements as well as embodiments wherein the subject-matter which “comprises” specifically listed elements may and/or indeed does encompass further elements. According to one embodiment, subject matter described herein as comprising certain steps in the case of methods or as comprising certain ingredients in the case of compositions, solutions and/or buffers refers to subject matter consisting of the respective steps or ingredients.

The numbers given herein, in particular the relative amounts of a specific glycosylation property, are preferably to be understood as approximate numbers. In particular, the numbers preferably may be up to 10% higher and/or lower, in particular up to 9%, 8%, 7%, 6%, 5%, 4% 3%, 2% or 1% higher and/or lower. According to one embodiment, the numbers given herein are to be understood as exact numbers which may not be higher or lower.

This invention is not limited by the exemplary methods and materials disclosed herein. Numeric ranges are inclusive of the numbers defining the range. The headings provided herein are not limitations of the various aspects or embodiments of this invention which can be read by reference to the specification as a whole. It is preferred to select and combine preferred embodiments described herein and the specific subject-matter arising from a respective combination of preferred embodiments also belongs to the present disclosure.

### FIGURES

FIG. 1 shows the relative change of the mean follicle size in healthy female volunteers after a single administration of the indicated amount of FSH (invention). The follicle size before administration is used as reference (100%).

FIG. 2 shows the relative change of the mean follicle size in healthy female volunteers after a single administration of placebo or 150 IU Bravelle or Gonal-f. The follicle size before administration is used as reference (100%).

FIG. 3 shows the relative change of the mean follicle size as an average of all subjects after a single administration of the indicated amount of FSH (invention) (A) or placebo, 150 IU Bravelle or 150 IU Gonal-f (B). The follicle size before administration is

FIG. 4 shows the concentration of FSH in the serum of healthy female volunteers during a multiple dose study with daily administration of FSH (invention) (also administration every 2^{nd} day), Gonal-f or Bravelle. Similar amounts of FSH administered resulted in comparable FSH serum levels.

FIG. 5 shows the mean number of follicles with a diameter of 8.0 mm or more observed in healthy female volunteers (mean of 10 subjects) after daily dosing of 150 IU FSH (invention) for 7 days. The coloring of the bars indicates the follicle size.

FIG. 6 shows the mean number of follicles with a diameter of 8.0 mm or more observed in healthy female volunteers (mean of 10 subjects) after daily dosing of 150 IU Gonal-f for 7 days. The coloring of the bars indicates the follicle size.

FIG. 7 shows the mean number of follicles with a diameter of 8.0 mm or more observed in healthy female volunteers (mean of 10 subjects) after daily dosing of 150 IU Bravelle for 7 days. The coloring of the bars indicates the follicle size.

FIG. 8 shows the mean number of follicles with a diameter of 8.0 mm or more observed in healthy female volunteers (mean of 10 subjects) after daily dosing of 75 IU FSH (invention) for 7 days. The coloring of the bars indicates the follicle size.

FIG. 9 shows the mean number of follicles with a diameter of 8.0 mm or more observed in healthy female volunteers (mean of 10 subjects) after administration of 150 IU FSH (invention) every second day for 7 days. The coloring of the bars indicates the follicle size.

FIG. 10 shows the concentration of inhibin-B (A) and estradiol (B) in the serum of healthy female volunteers after a multiple dose study with administration of FSH (invention) (75 IU daily, 150 IU daily, 150 IU every 2^{nd} day) or Gonal-f (150 IU daily) during days 1 to 7.

FIG. 11 shows the mean number of follicles with a diameter of 10.0 mm or more observed in healthy female volunteers (mean of 10 subjects) after administration of (A) 150 IU FSH (invention), (B) 150 IU Gonal-f or (C) 75 IU FSH (invention) daily or (D) 150 IU FSH (invention) every second day for 7 days. The coloring of the bars indicates the follicle size.

FIG. 12 shows the mean plasma FSH concentration versus time after the last of multiple doses of subcutaneously administered FSH.

FIG. 13 shows the cAMP release of isolated granulosa cells stimulated with different concentrations of the improved recombinant human FSH (FSH (invention); preparation 1: open squares, preparation 2: closed triangles) or FSH obtained from CHO cells (Gonal F; closed diamonds).

FIG. 14 shows the estradiol synthesis of isolated granulosa cells stimulated with different concentrations of the improved recombinant human FSH (FSH (invention); preparation 1: open squares, preparation 2: closed triangles) or FSH obtained from CHO cells (Gonal F; closed diamonds).

FIG. 15 shows the progesterone synthesis of isolated granulosa cells stimulated with different concentrations of the improved recombinant human FSH (FSH (invention); preparation 1: open squares, preparation 2: closed triangles) or FSH obtained from CHO cells (Gonal F; closed diamonds).

FIG. 16 shows the cAMP release of isolated granulosa cells stimulated with different concentrations of the improved recombinant human FSH (FSH (invention); open squares) or urinary FSH (Fostimon; closed diamonds).

FIG. 17 shows the estradiol synthesis of isolated granulosa cells stimulated with different concentrations of the improved recombinant human FSH (FSH (invention); open squares) or urinary FSH (Fostimon; closed diamonds).

FIG. 18 shows the progesterone synthesis of isolated granulosa cells stimulated with different concentrations of the improved recombinant human FSH (FSH (invention); open squares) or urinary FSH (Fostimon; closed diamonds).

FIG. 19 shows the results of the Steelman-Pohley assay using the improved recombinant human FSH in comparison to standard urinary FSH and standard recombinant FSH obtained from CHO cells. The ovarian weight gain in immature female rats after daily administration for three days is plotted against the used FSH concentration.

FIG. 20 shows schematic drawings of complex-type glycan structures which may be attached to the FSH glycosylation sites. Shown are (a) biantennary, (b) triantennary and (c) tetraantennary structures. One or more of the sialic acid and galactose residues may also be absent in these structures and the structures may further comprise, for example, a bisecting GlcNAc residue, a fucose residue and/or sulfate groups. Sia: sialic acid; Gal: galactose, also referred to herein as terminal galactose; GlcNAc: N-acetylglucosamine; Man: mannose.

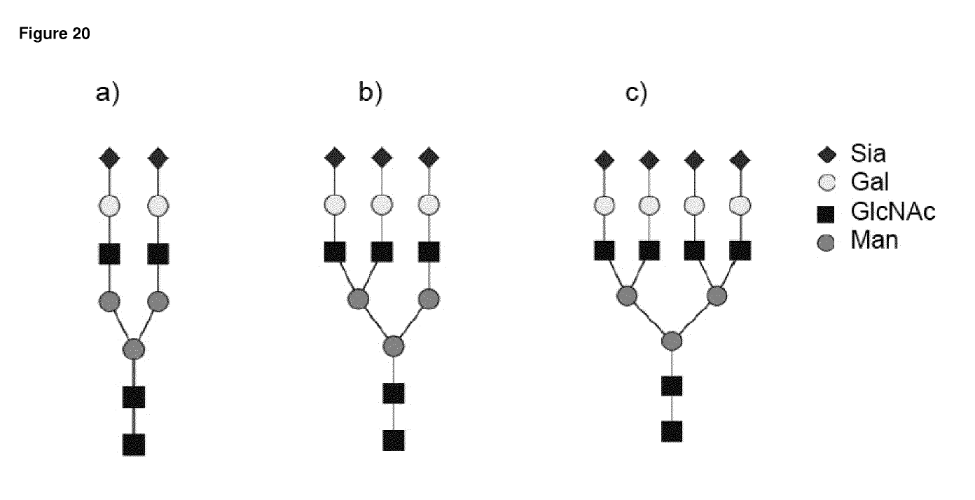

### EXAMPLES

### Example 1

### Preparation of FSH (Invention)

FSH is produced by cultivation of GT-5s cells stably transfected with two expression constructs harbouring the alpha and beta chain of human FSH (alpha chain accession number NT_007299.13; beta chain accession number NT_{1'}009237.18). The plasmid for the expression of the FSH alpha chain is carrying the gene of a mutated version of the murine dihydrofolate reductase (dhfr) with higher resistance to the enzyme inhibitor methotrexate than the native form and the second plasmid for the expression of the FSH alpha chain is carrying the puromycin resistance gene.

Transfection of the cell line for FSH (invention) expression was performed by nucleofection using the two expression plasmids described above. For selection and amplification of stable antibody producing cell clones puromycin and methotrexate were added at increasing concentrations. Amplified cell pools were seeded in a semi-solid matrix for single cell cloning by the Clone PixFL technology or single cell cloning by limited dilution. The clones were screened for high secretion of intact FSH molecules.

FSH is produced by fermentation of the final FSH producing GT-5s clone in batch, fed-batch or perfusion process under serum free conditions. The fermentation is usually run for 2-3 weeks.

After fermentation the supernatant is filtered through 2 μm filters to eliminate cells and cell debris prior to a sterile filtration step using 0.2 μm filters. The purification process utilizes a reverse phase chromatography (RPC) as capture step followed by a concentration step and a subsequent size exclusion chromatography (SEC). Optionally, the eluate is then applied to an anion exchange chromatography (AEC) to eliminate the less acidic FSH contents. This is done by washing the bound FSH with washing buffer at pH 5.0 (“enrichment at pH 5.0”) or pH 4.5 (“enrichment at pH 4.5”) to elute less acidic FSH isoforms prior to elution of the desired FSH fraction. As a polishing step a hydrophobic interaction chromatography (HIC) is used to gain FSH at high purity.

### Example 2

### Phase II Clinical Studies with FSH (Invention)

A phase II clinical study with FSH (invention) and a comparator agent (Gonal-F) was performed to investigate the therapeutic efficacy and safety of various dosages of the FSH preparations.

240 randomized female human patients with indication for intracytoplasmatic sperm each comprising 40 patients, the patients were treated with 52.5 IU, 75 IU, 112.5 IU or 150 IU FSH (invention) per day, 150 IU FSH (invention) every second day or 150 IU Gonal-f per day.

The treatment cycle for each patient included down-regulation of the endogenous hormone level using a GnRH agonist protocol, stimulation of follicle growth by administration of FSH, retrieval of the oocytes, ICSI and embryo transfer. Stimulation with FSH was done for up to 18 days until at least one follicle reached a diameter of at least 20 mm. Mean duration of FSH treatment was about 9 to 10 days. Then final maturation of the oocytes was induced by administration of a single dose of hCG about 1 day after the last FSH dose. 32 to 36 hours after hCG administration, all follicles having a size of at least 12 mm were punctured. 2 to 3 days after oocyte retrieval, selected oocytes were fertilized by ICSI and a maximum of two embryos per patient were transferred.

As result, stimulation of follicle growth with FSH (invention) led to a higher number of follicles and retrieved oocytes than with Gonal-f at half or three quarter the dose of Gonal-f:

TABLE 2
Comparison of FSH (invention) and Gonal-f

| | FSH (invention) | | | |
|---|---|---|---|---|
| | | 150 IU | | |
| | | every | | Gonal-F |
| Mean number of | 75 IU | 2^{nd} day | 112.5 IU | 150 IU |
| follicles ≧12 mm | 13.0 | 12.8 | 13.9 | 12.4 |
| (follicles large enough | (+5%) | (+3.2%) | (+12%) | |
| for puncture) | | | | |
| retrieved COCs | 12.6 | 13.4 | 14.4 | 11.1 |
| (obtained oocyte | (+13.5%) | (+20.7%) | (+29.5%) | |
| complexes) | | | | |
| retrieved metaphase II | 9.4 | 10.1 | 10.7 | 8.6 |
| oocytes | (+9.5%) | (+17.4%) | (+24.5%) | |
| (obtained mature oocytes) | | | | |
| 2PN oocytes (one day | 7.3 | 7.5 | 7.5 | 6.2 |
| after puncture) | (+17.5%) | (+21%) | (+21%) | |
| (oocytes fertilized by | | | | |
| ICSI (two core stadium)) | | | | |

The results show that FSH (invention), in comparison to Gonal-f, induces the development of a significantly increased number of large follicles even at lower doses (down to only 50% of the dose of Gonal-f). Likewise, also the number of retrieved cumulus-oocyte complexes (COCs), the number of metaphase II oocytes and the number of successfully fertilized oocytes are significantly higher for FSH according to the present invention when compared to Gonal-f at a up to 2-fold higher dose. This impressively demonstrates the superior activity of FSH (invention).

Furthermore, also the relative number of successful fertilizations based on the identified developed follicles is increased for FSH (invention). For example, in the patients treated with 150 IU FSH (invention) every second day, 59% of all follicles with a diameter of at least 12 mm which were identified in the treated patients were successfully fertilized and formed a two core oocyte (2PN). In contrast, only 50% of the follicles 12 mm identified in the patients treated with Gonal-f were successfully fertilized. Hence, the follicles induced by FSH (invention) demonstrated a higher quality than those induced by Gonal-f.

### Example 3

### Phase I Clinical Studies with FSH (Invention)

A phase I clinical study with FSH (invention) and comparator agents (Gonal-F and Bravelle) was performed to determine the therapeutic efficacy of the FSH preparations.

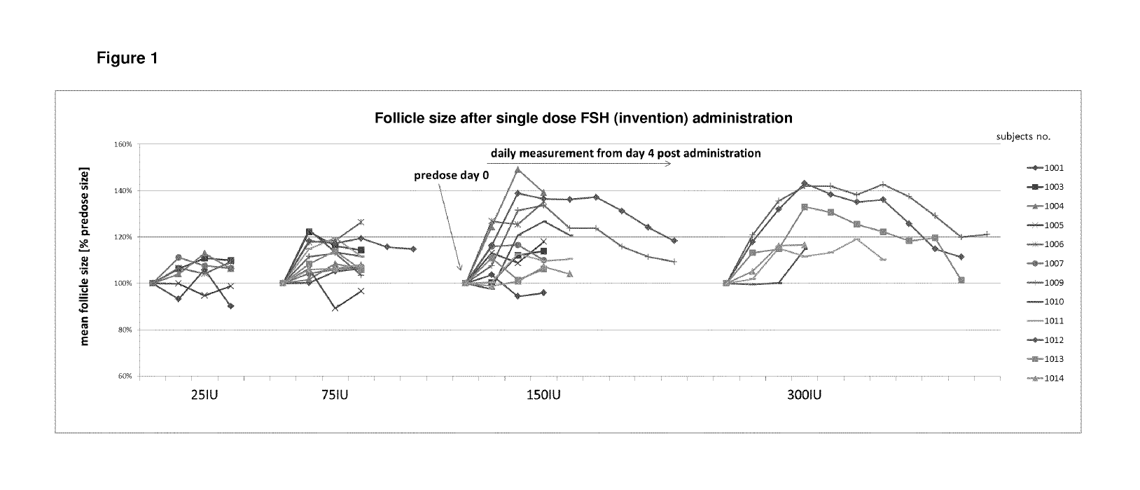

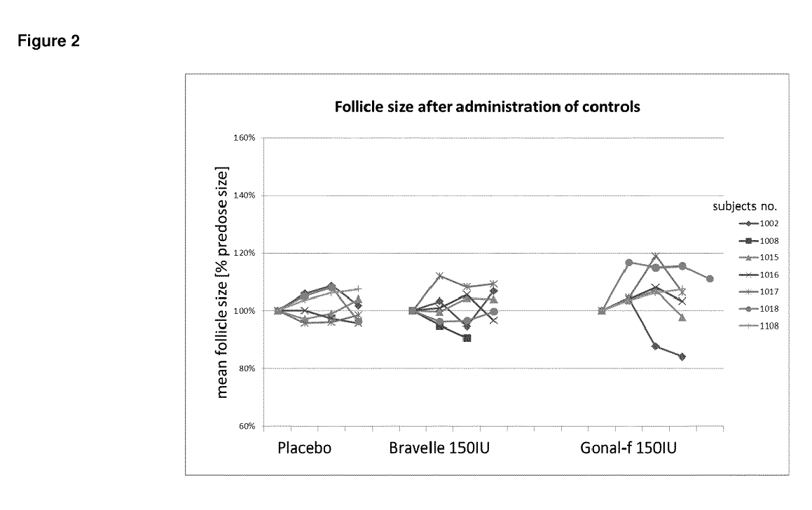

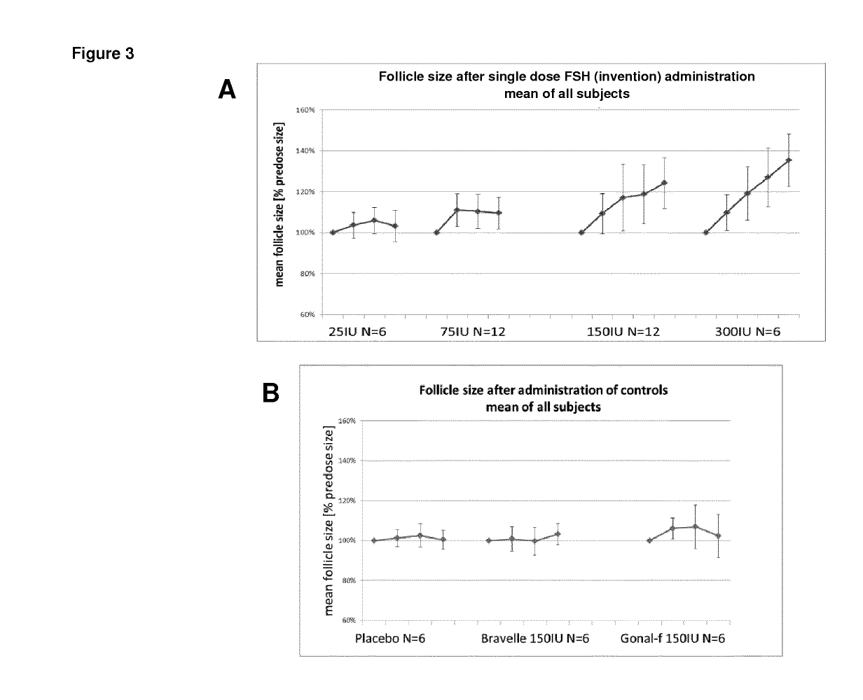

FSH was administered to female volunteers and the pharmacokinetic and pharmacodynamic parameters were determined. In a first study, the healthy female volunteers received 25 IU, 75 IU, 150 IU or 300 IU FSH (invention) in a single subcutaneous dose and the mean follicle size in relation to the pre-dose size was determined by daily measurements from day 4 post administration. As control, volunteers received placebo or 100 IU Bravelle or Gonal-F. As shown in FIGS. 1, 2 and 3, the mean follicle size significantly increased after a single dose of FSH (invention). Increase in follicle size was dose dependent. The increase in follicle size was significantly greater when compared to placebo or the reference FSH preparations (Bravelle and Gonal-F). Hence, it was shown that FSH (invention) has a much higher potency of inducing follicular growth than the commonly used FSH preparations and is capable of inducing significant follicular growth even after a single dose.

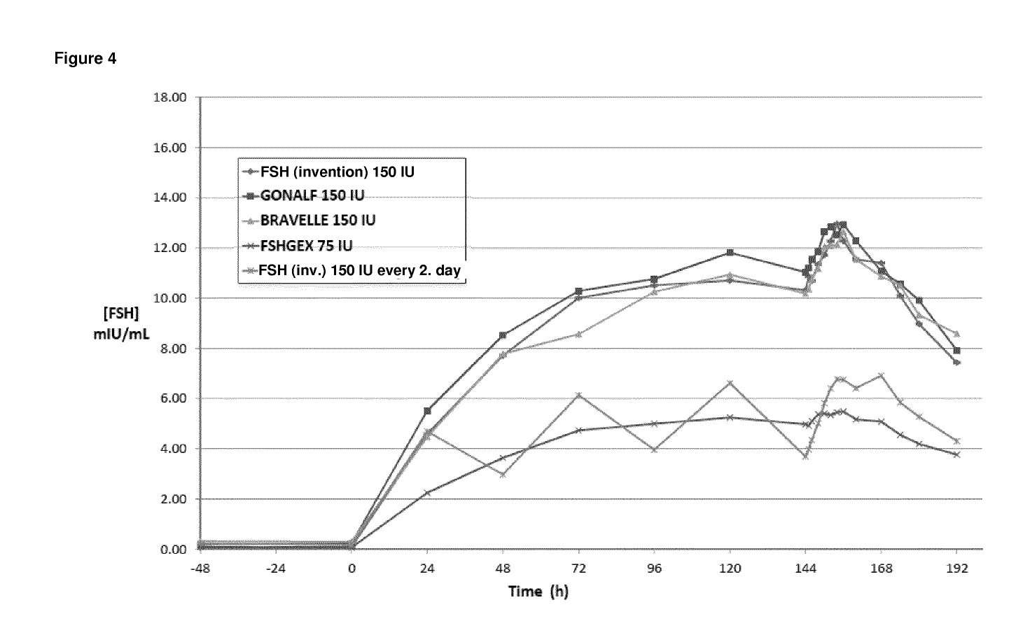

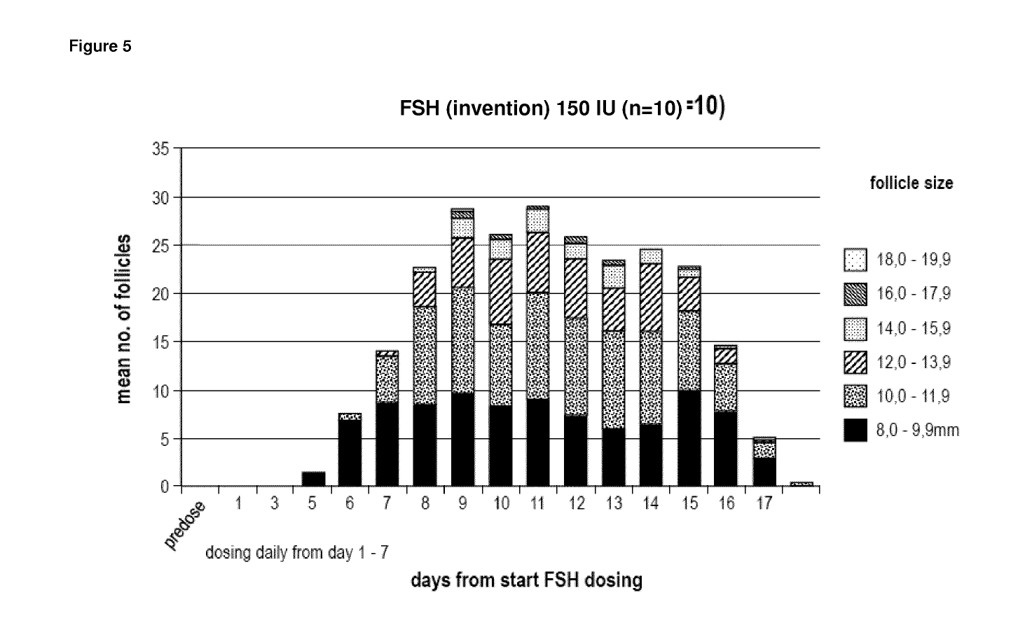

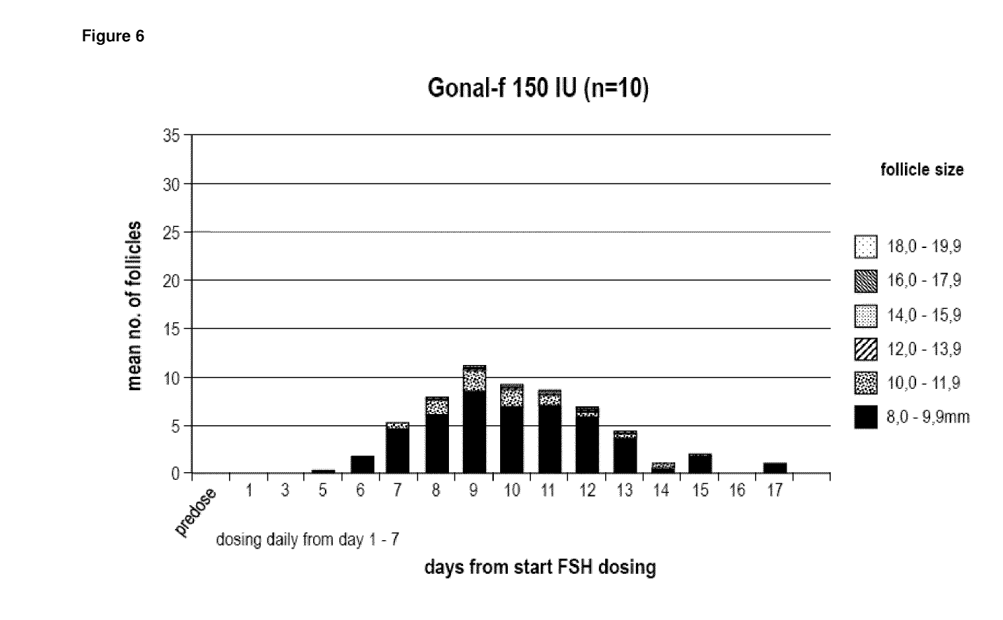

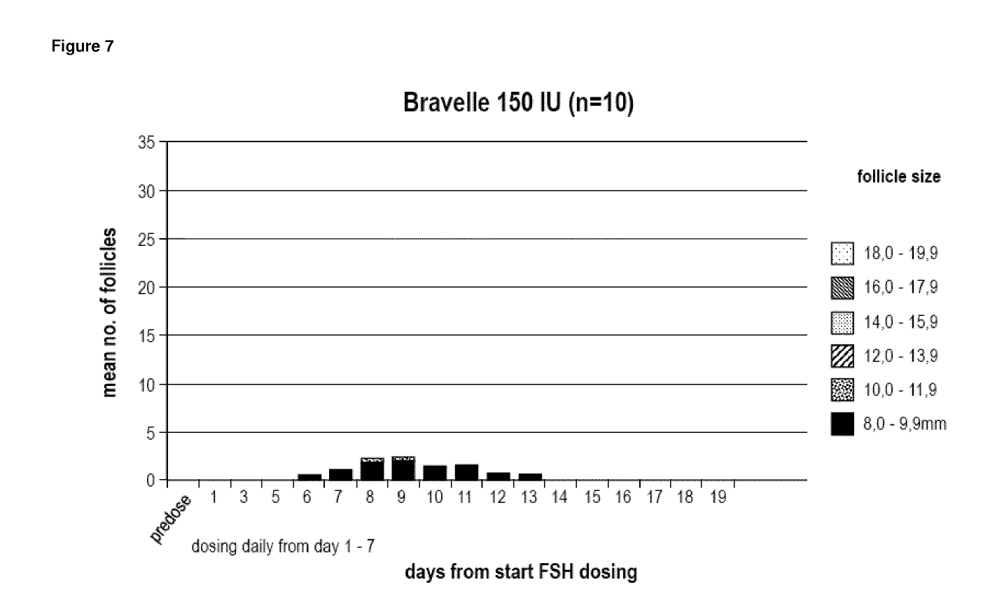

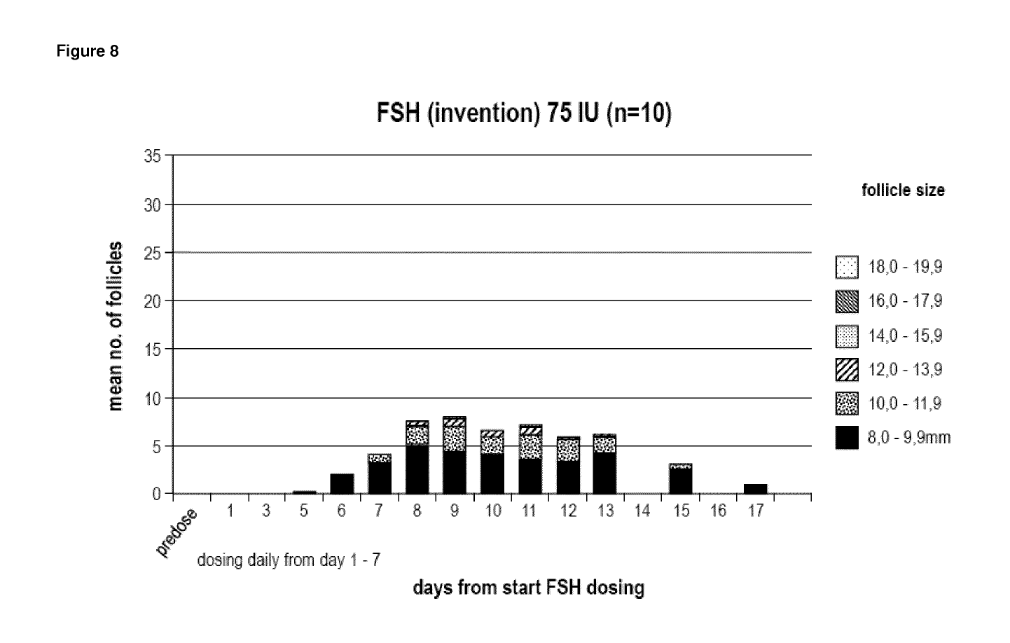

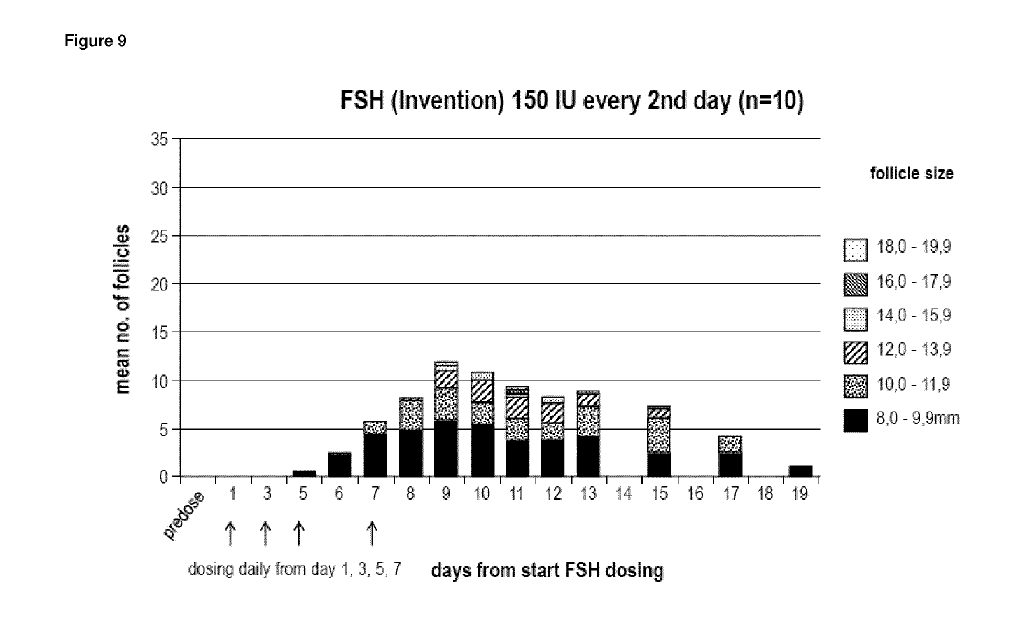

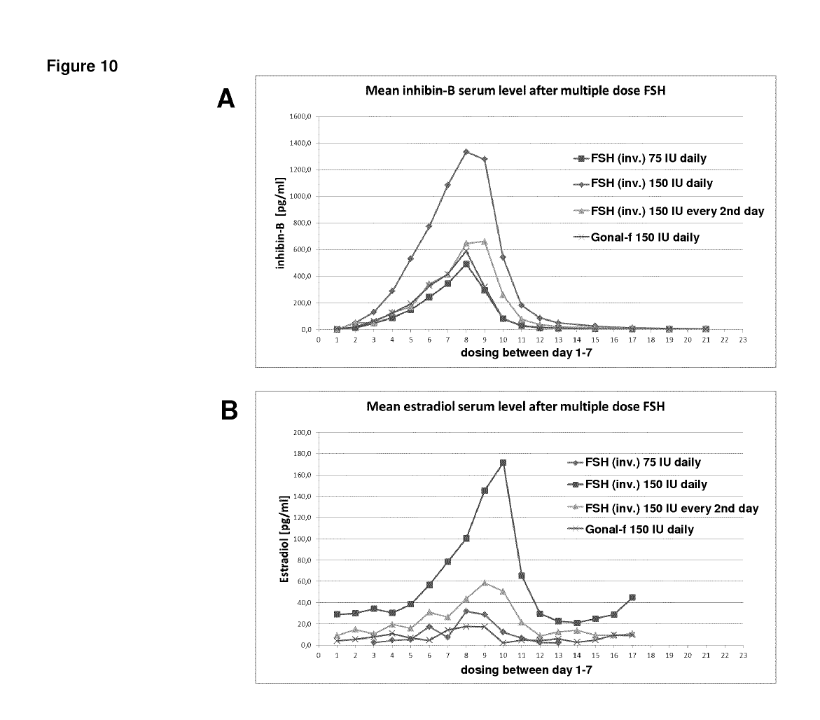

Furthermore, a multiple dose clinical study was performed. FSH (invention), Gonal-F and Bravelle were administered with daily doses of 150 IU for seven days. In a further cohort, FSH (invention) was given at daily doses of 75 IU for seven days. In another cohort, FSH (invention) was administered every second day in a dose of 150 IU. FSH was administered after down-regulation of the menstruation cycle, resulting in the stimulation of follicle growth. Serum levels of FSH, inhibin-b and estradiol were monitored and the number and size of the follicles were determined. As shown in FIG. 4, the serum levels of FSH were comparable for the different FSH preparations administered at equal doses. FSH (invention) given at half dose or every second day resulted in halving of the FSH serum level, with the administration every second day showing the expected fluctuation (see FIG. 4). However, as shown in FIGS. 5 to 7, the administration of FSH (invention) results in a markedly increased number and size of induced follicles compared to Gonal-F and Bravelle. Administered at half dose, FSH (invention) results in a follicular growth comparable to that of Gonal-F (see FIG. 8). Furthermore, the administration of FSH (invention) every second day results in a comparable number, but markedly increased size of the induced follicles when compared to Gonal-F administered with the same dose, but every day instead of every second day (see FIG. 9). A similar increase in follicle size can also be seen when comparing the administration of 150 IU FSH (invention) every second day and 75 IU FSH (invention) every day, which results in the same amount of FSH administered. Hence, the same amount of FSH (invention) can result in a much increased follicular size when given every second day rather than every day. This difference is also observed in inhibin-b and estradiol levels in the patient serum, wherein FSH (invention) given every second day at 150 IU shows a significantly increased level compared to FSH (invention) administered every day at 75 IU or Gonal-F administered every day at 150 IU (see FIG. 10).

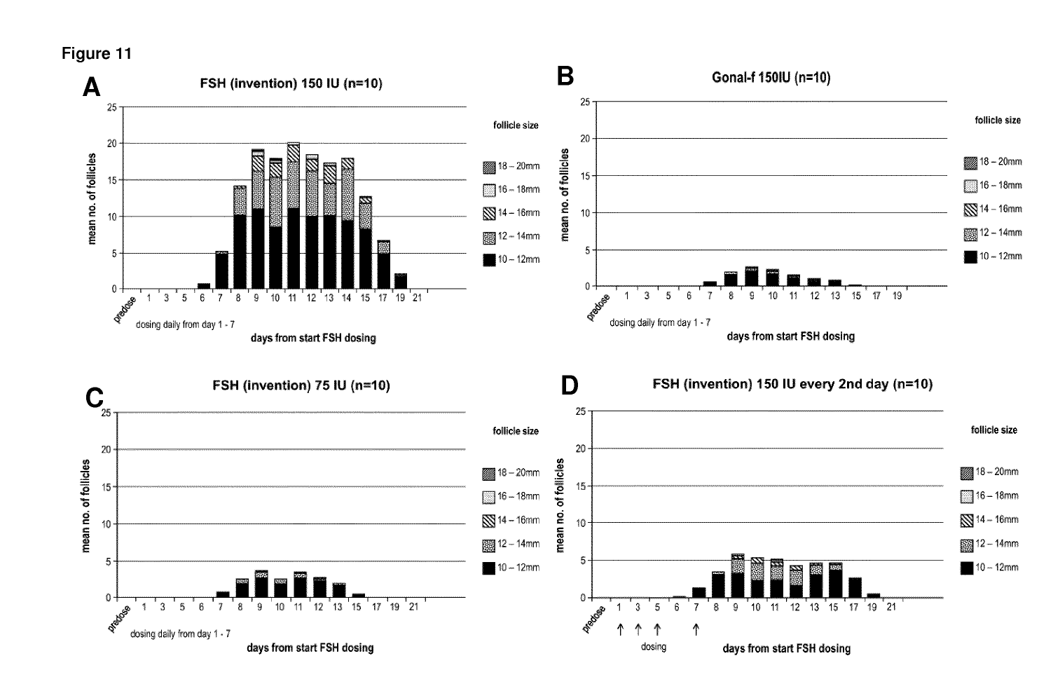

In addition, the high number of follicles induced with FHS (invention) is observed for several days in the patients. In particular The number of follicles having a size of at least 10 mm is maintained in the patient treated with FSH (invention) on average for about 5 days (see FIG. 11). For example, the number of large follicles is essentially constant throughout days 8/9 to 14/15, i.e. after termination of the FSH administration. In contrast, Gonal-f shows a peak in the number of large oocytes on days 9 and 10, which thereafter rapidly declines. Hence, FSH (invention) shows a trailing effect, maintaining the developmental status of the follicles for several days after termination of the FSH application. This effect is important for infertility treatment as it broadens the window for a successful induction of the final oocyte maturation. In common treatments, hCG or other ovulation inducers have to be administered to the patient about 1 day after termination of the FSH administration. Else, the induced large follicles will regress and final maturation of the oocytes is no longer possible. With FSH (invention), the oocytes stay significantly longer, i.e. about 5 to 6 days, at the achieved size and maturation status after termination of the FSH administration. Hence, stimulation of the final oocyte maturation and induction of ovulation, using, e.g. hCG, is possible for a much longer time interval when using FSH (invention) for stimulation of follicle growth compared to the use of Gonal-f.

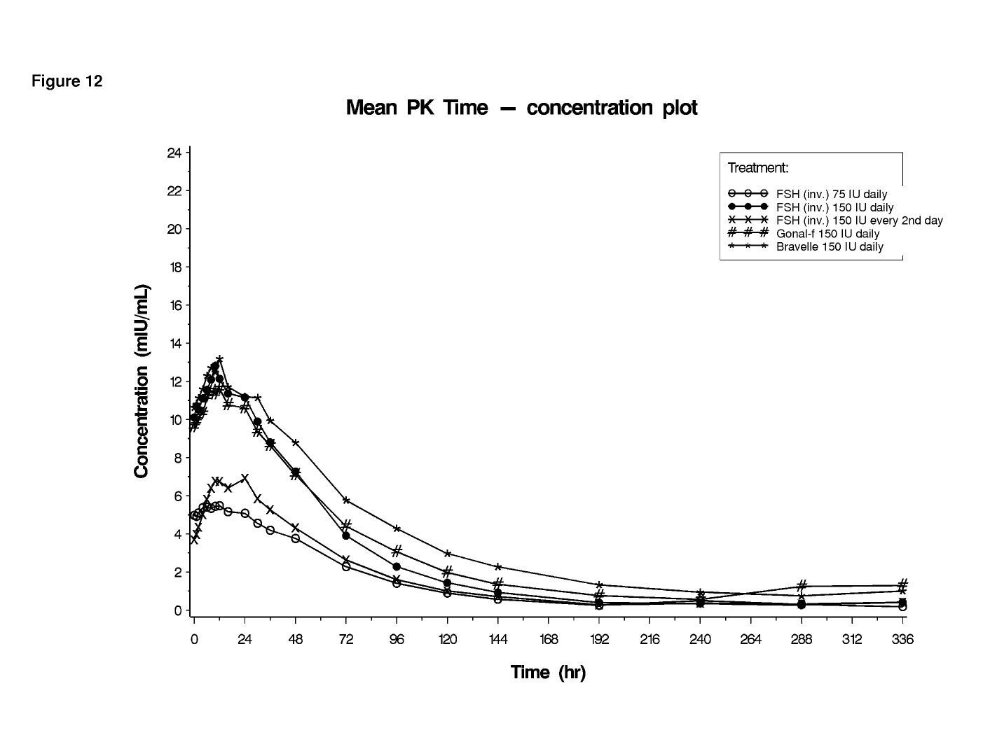

Furthermore, the pharmacokinetic of FSH was analyzed after the above-described multiple dosage regimen. For this, the serum level of FSH was monitored following the multiple dose administration. FIG. 12 shows the mean concentration versus time curves for plasma FSH after the last injection of multiple subcutaneous injections of 75 or 150 IU FSH (invention) administered daily and 150 IU FSH (invention) administered every second day, 150 IU Bravelle and 150 IU QD Gonal-f administered daily on a linear scale. The plots show an increase in the plasma FSH concentration after the last subcutaneous injection of multiple injections. After the peak plasma FSH concentration (Cₘₐₓ) the plasma FSH concentration decreased to baseline level. The Cₘₐₓ of plasma FSH increased with an increasing dose level of FSH (invention). Cₘₐₓ decreased when 150 IU FSH (invention) was administered once every two days instead of once daily. When comparing the concentration versus time plots (FIG. 12) of 150 IU FSH (invention administered daily with the same dose levels of the comparators Bravelle and Gonal-f, it can be seen that the curves are highly similar. The Cₘₐₓ after 150 IU FSH (invention) (12.989 mIU/mL), Bravelle (13.370 mIU/mL), and Gonal-f (12.281 mIU/mL) were comparable. The AUC₀₋ₗₐₛₜ of Bravelle (1172.066 h*mIU/mL) was higher than the AUC₀₋ₗₐₛₜ of FSH (invention) (824.897 h*mIU/mL) and Gonal-f (917.400 h*mIU/mL). Also the circulation half-life t₁₁₂ of the different FSH preparations was comparable, with that of Bravelle being slightly higher (FSH (invention): ˜33 h; Gonal-f: ˜36 h; Bravelle: ˜54 h). This is remarkable as Bravelle showed a significantly lower pharmaceutical efficacy (see above).

In conclusion, it was demonstrated in the phase I clinical study that FSH (invention) has a much higher therapeutic efficacy in terms of follicular growth compared to the same amount of Gonal-F and Bravelle. Furthermore, a dosage regimen wherein FSH (invention) is administered every second day results in markedly increased follicular size compared to administration every day.

### Example 4

### Granulosa Cell Assay

In order to perform a granulosa cell assay primary cells are isolated from the follicular fluid of IVF patients during the collection of the oocytes. After a Ficoll gradient centrifugation which eliminates other cell types as e.g. red blood cells the granulosa cells are seeded in 24 to 96 well plate format for 5-7 days in culture medium containing androstendione or testosterone. After that period, the cells (2 to 4*10⁴ cells per well) are stimulated with FSH ranging between 1 pg/ml to 2 μg/ml in the steps shown in the diagram (400 μl medium per well). After three to four hours incubation half of the supernatant is collected for performing the cAMP assay. Another 24 h later the cells are lysed by freeze thaw in the remaining supernatant. The lysate is applied in the progesterone and estradiol assays.

Comparison of FSH (invention) and Gonal F

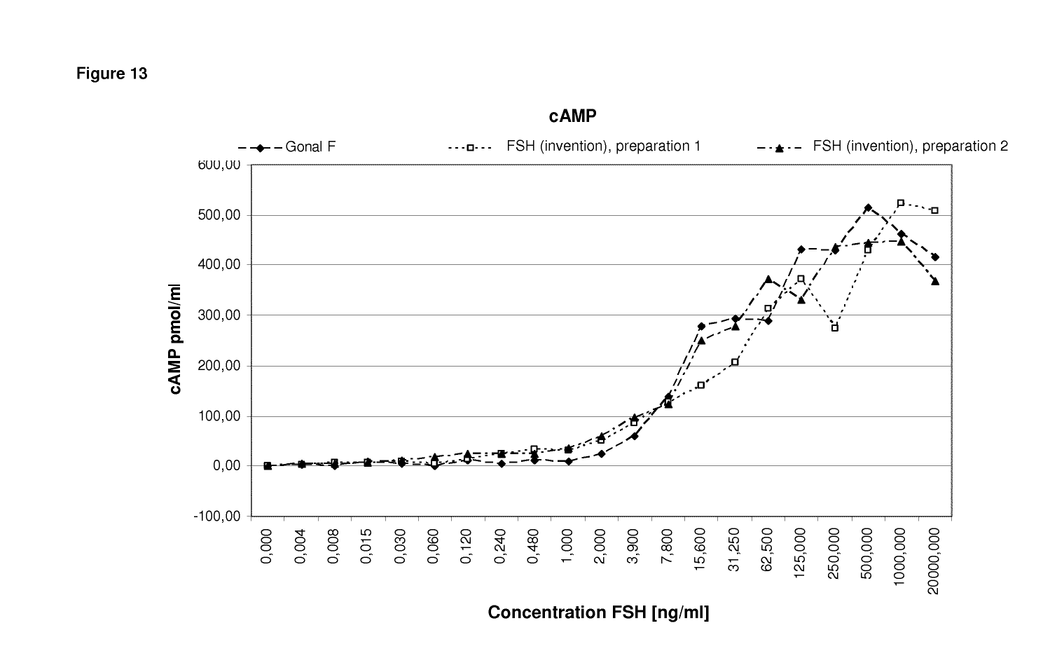

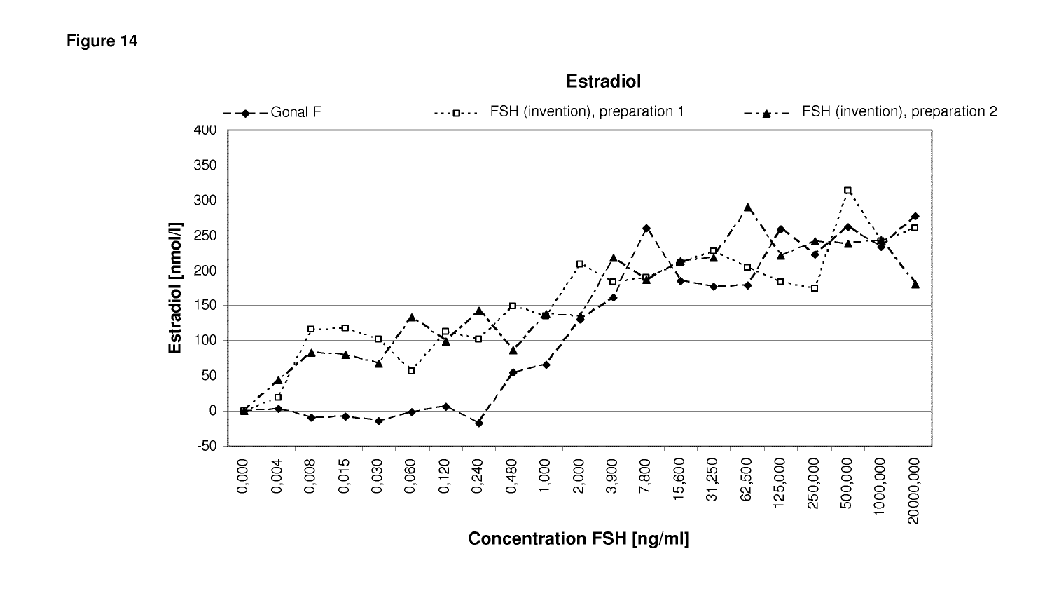

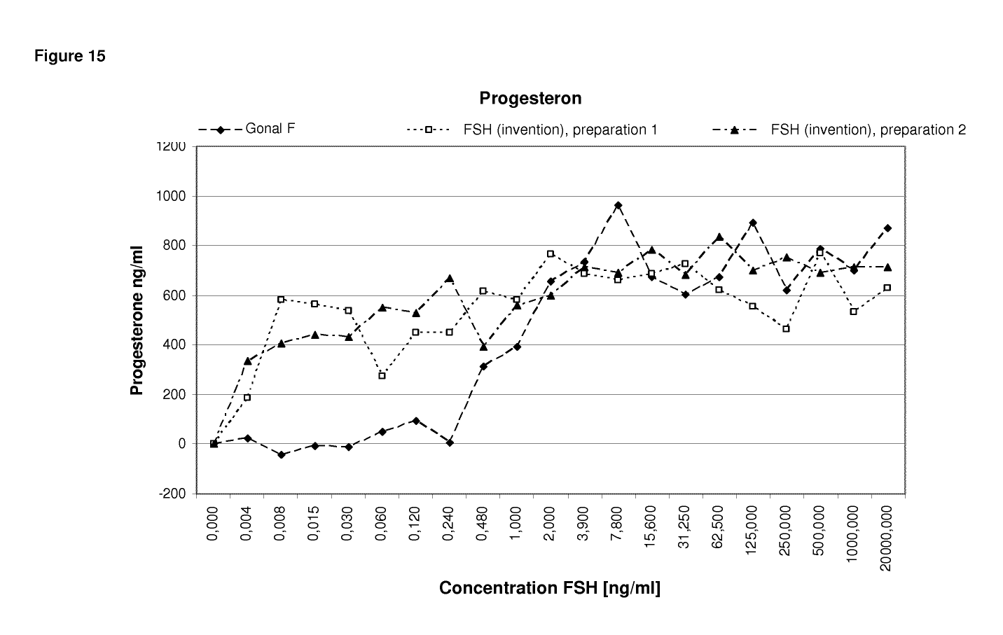

In the first set of experiments FSH (invention) is compared to Gonal F (Merck Serono SA). Gonal F is FSH recombinantly produced in CHO cells. The results are shown in FIGS. 13 to 15. While the second messenger cAMP is produced at comparable FSH concentrations of Gonal F and FSH (invention) products in comparable amounts, the steroids progesterone and estradiol are released at much lower FSH concentrations in the case of FSH (invention) products compared to FSH recombinantly produced in CHO cells (Gonal F).

Comparison of FSH (invention) and Fostimon

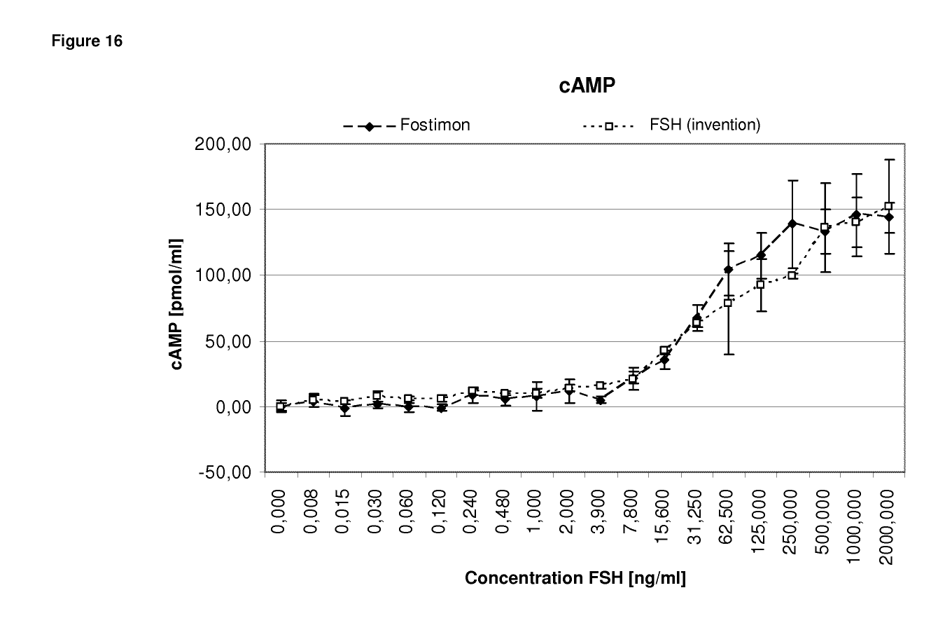

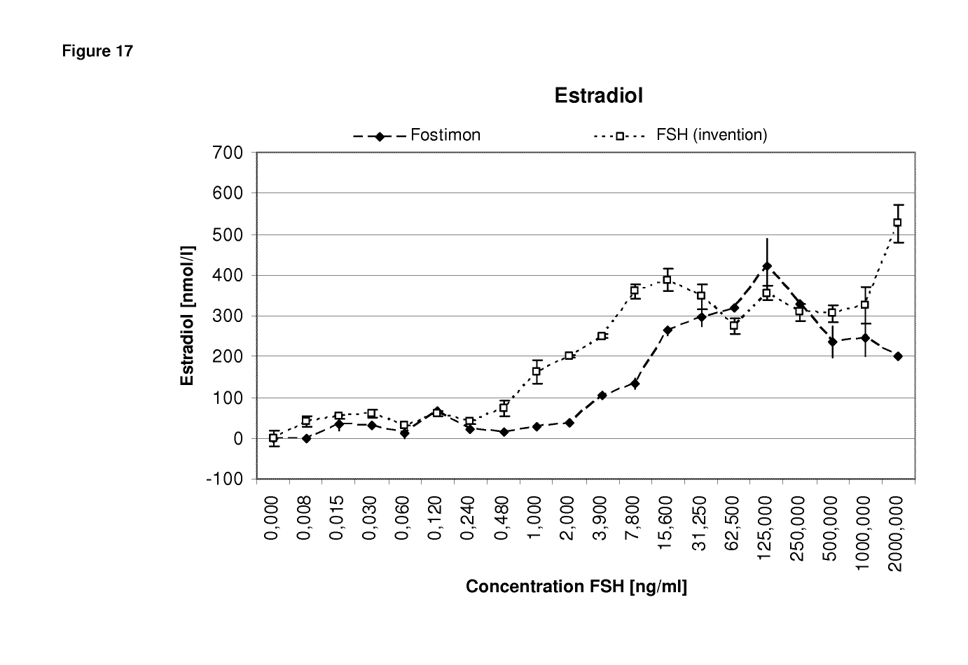

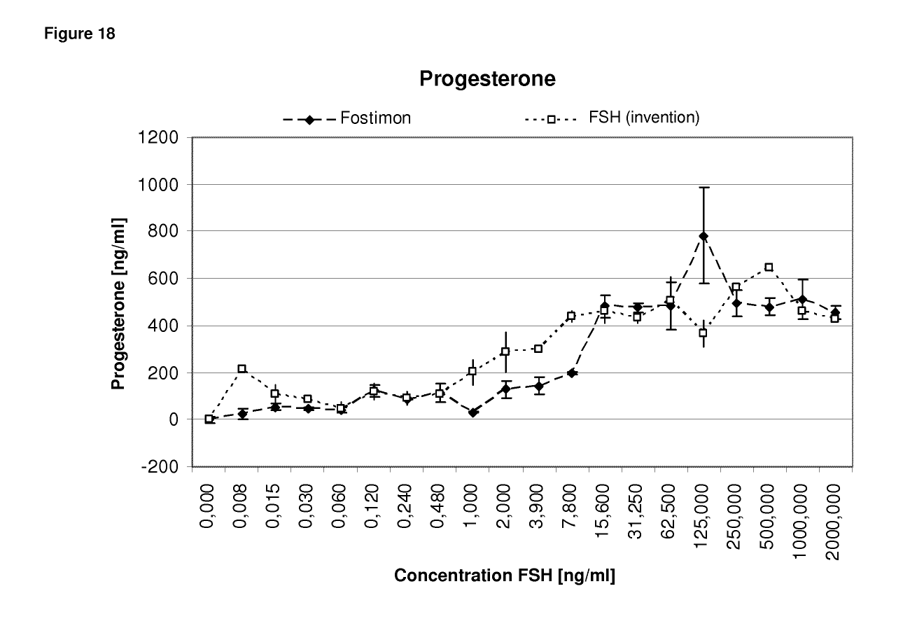

In another set of experiments the FSH (invention) was compared against Fostimon (IBSA Institut Biochimique SA), the FSH product isolated out of human urine. The results are shown in FIGS. 16 to 18. While the cAMP level rises similarly at comparable dose ranges of FSH for both products, the sex steroids are produced at a significantly lower concentration of FSH (invention) compared to Fostimon.

Note: Since the assays are performed using different donors, differences in the stimulation profile may account to the donors used in each assay.

### Example 5

### Steelman-Pohley Assay

The activity of FSH was also determined by the Steelman-Pohley assay. The assay was performed according to the pharmacopeia. In particular, the ovarian weight gain in immature female rats was measured after administration of three different FSH concentrations each given daily for three days. The potency is calculated using the parallel line evaluation. The Steelman-Pohley assay was used to determine the standard international units (IU) of the FSH preparations according to the invention.

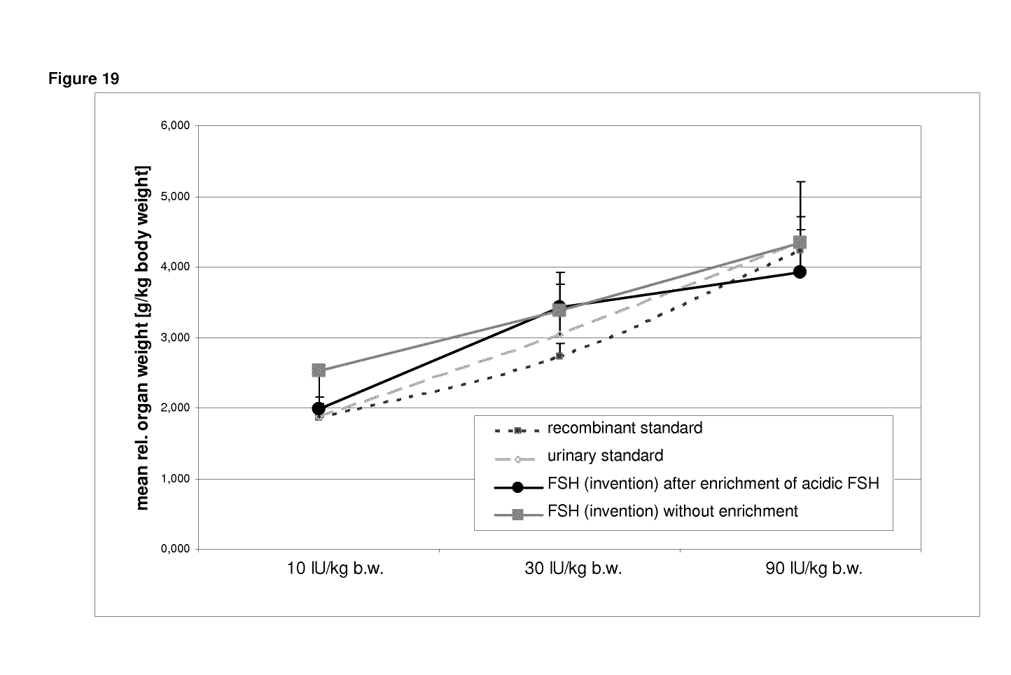

As demonstrated by the Steelman-Pohley assay, the in vivo activities of the FSH (invention) and of the urinary and recombinant standard FSH are similar in rat (see FIG. 19).

### Example 6

### Glycoprofiling

The glycoprofiles of the different FSH preparations were determined by structural analysis of the glycosylation. Glycoprofiling generates information on the complex glycan structure of the glycosylation sites. For glycoprofiling, the intact N-glycans were released from the protein core and the reducing ends of N-glycans were labeled with a fluorescence marker. The purified sample of the labeled N-glycans was separated by UPLC. Peak areas based on fluorometric detection were employed for calculation of the relative molar abundances of the N-glycan structures. Estimated data for the FSH are summarized in Table 3. The values represent the relative molar contents of N-glycans containing the interesting type of monosaccharide (e.g. fucose).

TABLE 3
Relative amounts of the different glycosylation properties

| Sample | F | S | S0 | S1 | S2 | S3 | S4 | G | B | Sulf |
|---|---|---|---|---|---|---|---|---|---|---|
| FSH (invention) | 38% | 97% | 2% | 21% | 43% | 19% | 14% | 99% | 34% | 9% |
| Fostimon | 48% | 83% | | | | | | 91% | 28% | |
| Puregon¹ | 29% | 91% | | | | | | 91% | 0% | |

F: glycans containing fucose;
S: glycans containing at least one sialic acid;
S0: glycans containing no sialic acid;
S1: glycans containing one sialic acid;
S2: glycans containing two sialic acids;
S3: glycans containing three sialic acids;
S4: glycans containing four sialic acids;
G: glycans containing galactose;
B: glycans containing bisecting N-acetylgalactosamine;
Sulf: sulfated N-glycans
¹literature values (Hård, K. et al. (1990) European Journal of Biochemistry 193, 263-271)

Shown are the relative amounts of N-glycans on the FSH which carry the indicated units. Puregon is another recombinant human FSH produced in CHO cells.

Furthermore, the ratio of 2,3-coupled and 2,6-coupled sialic acids in the glycans of the FSH was analyzed by comparing the amount of sialic acid released by sialidase A (cleaving off 2,3- and 2,6-coupled sialic acids) and sialidase S (cleaving off only 2,3-coupled sialic acids).

TABLE 4
Relative amounts of the sialic acid linkage

| Sample | 2,3-linked sialic acid | 2,6-linked sialic acid |
|---|---|---|
| FSH (invention) | 43% | 57% |
| Bravelle | 75% | 25% |
| Gonal F/Puregon | 100% | 0% |

In FSH (invention), the sialic acid residues are coupled to the glycans by 2,3- as well as 2,6-bonds in a ratio of about 1:1, comprising even more 2,6-coupled sialic acids than 2,3-coupled sialic acids, while in the urinary FSH Bravelle (Ferring Pharmaceuticals Inc.) the ratio is about 3:1 in favor of 2,3-linked sialic acid. Due to their recombinant production in CHO cells, Puregon (Organon/EssexPharma) and Gonal F (Merck Serono) do not have any bisecting N-acetylgalactosamines and only comprises 2,3-coupled sialic acids.

Antennarity, terminal galactose units and Z-number were calculated from the above measurements and by determination of the charge distribution of the glycans after release from the FSH.

TABLE 5
Antennarity of the glycosylation of the different FSH

| Sample | Bi | Tri | Tetra |
|---|---|---|---|
| FSH (invention) | 51% | 26% | 21% |
| Fostimon | 39% | 45% | 16% |
| GonalF¹ | ~65% | ~25% | ~10% |
| Puregon² | 53% | 26% | 12% |

Bi: biantennary N-glycans;
Tri: triantennary N-glycans;
Tetra: tetraantennary N-glycans
¹literature values (Gervais, A. et al. (2003) Glycobiology 13(3), 179-189)
²literature values (Hård, K. et al. (1990) European Journal of Biochemistry 193, 263-271)

Shown are the relative amounts of bi-, tri- and tetraantennary N-glycans on the FSH.

TABLE 6
Z-number of different FSH

| Sample | Z-number |
|---|---|
| FSH (invention) | 220 |
| Gonal F (rFSH) | 218 |
| Puregon (rFSH) | 204 |
| Fostimon (uFSH) | 212 |
| Bravelle (uFSH) | 244 |

Shown is the Z-number, i.e. the relative acidity, of the FSH preparations. A higher Z-number indicates a more acidic FSH preparation.

In conclusion, the FSH according to the present invention (FSH (invention)) has a high degree of bisecting N-acetlyglucosamine, a high antennarity, a high degree of sialylation and a high sulfation degree. It is assumed that because of one or more of these three glycosylation parameters, the FSH (invention) has a superior activity compared to the common recombinant or urinary FSH preparations.

Furthermore, the FSH (invention) has a ratio of 2,3- to 2,6-sialylation of about 1:1 or even a higher amount of 2,6-sialylation.

Furthermore, the glycan structures of the FSH preparations were also analyzed by mass spectroscopy of the released glycans. The following results were obtained:

TABLE 7
Relative amounts of different glycosylation properties

| Sample | F | S0 | S1 | S2 | S3 | S4 | S > 0 | G0 | G1 | G2 | G3 | G4 | G > 0 | B |
|---|---|---|---|---|---|---|---|---|---|---|---|---|---|---|
| Gonal F | 55 | 1 | 16 | 45 | 28 | 9 | 98 | 0 | 1 | 55 | 30 | 14 | 100 | 0 |
| Bravelle | 43 | 1 | 11 | 45 | 34 | 9 | 99 | 0 | 7 | 39 | 39 | 14 | 99 | 14 |
| FSH (inv.) | 43 | 1 | 18 | 35 | 31 | 15 | 99 | 0 | 7 | 45 | 30 | 20 | 102 | 28 |

shown are the relative amounts of glycans having the following property:
F: fucose;
S0: no sialic acid;
S1: one sialic acid;
S2: two sialic acids;
S3: three sialic acids;
S4: four sialic acids;
S > 0: at least one sialic acid;
G0: no galactose;
G1: one galactose;
G2: two galactoses;
G3: three galactoses;
G4: four galactoses;
G > 0: at least one galactose;
B: bisecting GlcNAc

TABLE 8
Antennarity of the glycosylation of the different FSH

| Sample | Bi | Tri | Tetra |
|---|---|---|---|
| FSH (invention) | 48% | 31% | 21% |
| Bravelle | 45% | 43% | 12% |
| Gonal-f | 56% | 30% | 14% |

Bi: biantennary N-glycans;
Tri: triantennary N-glycans;
Tetra: tetraantennary N-glycans

TABLE 9
Relative amount of sulfated glycans

| Sample | Sulfation |
|---|---|
| FSH (invention) | 15% |
| Bravelle | 2% |
| Gonal F | 0% |

Shown are the relative amounts of N-glycans on the FSH which carry a sulfate group.

1. A method for controlled ovarian hyperstimulation for stimulating the development of multiple ovarian follicles in a female subject, comprising (a) administering to a female subject a recombinant FSH preparation using a dosage regimen wherein the single doses sum up to an average amount of from about 35 to about 250 IU FSH per day;
(b) triggering ovulation when there are multiple follicles with a mean diameter equal to or greater than 12 mm and/or when there is at least one follicle with a diameter of at least 17 mm;
(c) obtaining multiple oocytes from the female subject, wherein on average at least 5 oocytes per female subject are obtained and/or at least 5 oocytes from the female subject are obtained;
wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation.

2. The method of claim 1, wherein
in step (a) a dosage regimen is used, wherein the single doses sum up to an average amount of from about 50 to about 125 IU FSH per day;
in step (b) ovulation is triggered when there is at least one follicle with a diameter of at least 17 mm;
in step (c) at least 5 oocytes are obtained from the female subject in the form of cumulus oocyte complexes (COCs), and at least 4 of these oocytes are metaphase II oocytes; and
wherein the recombinant FSH in the preparation has a glycosylation pattern comprising the following characteristics:
(i) a relative amount of glycans carrying bisecting N-acetylglucosamine (bisGlcNAc) in the range of from about 25% to about 50% of all glycans attached to the FSH in the preparation;
(ii) a relative amount of 2,6-coupled sialic acid in the range of from about 53% to about 80% of all sialic acid residues attached to the FSH in the preparation;
(iii) a relative amount of sulfated glycans of at least 5% of all glycans attached to the FSH in the preparation;
(iv) a relative amount of glycans carrying outer arm fucose of 5% or less of all glycans attached to the FSH in the preparation;
(v) a relative amount of glycans carrying core fucose of at least 30% of all glycans attached to the FSH in the preparation;
(vi) a relative amount of at least tetraantennary glycans of at least 16% of all glycans attached to the FSH in the preparation;
(vii) a relative amount of glycans carrying one or more sialic acid residues of at least 88% of all glycans attached to the FSH in the preparation; and
(viii) a Z number of at least 210.

3. The method of claim 1, wherein a dosage regimen is used in step (a), wherein the single doses sum up to an average amount of from about 50 to about 125 IU FSH per day.

4. The method of claim 1, wherein a dosage regimen is used in step (a), wherein about 50 to about 125 IU FSH are administered every day; or wherein about 100 to about 250 IU FSH are administered every second day; or wherein about 150 to about 375 IU FSH are administered every third day.

5. The method of claim 1, wherein a dosage regimen is used in step (a), wherein the single doses sum up to an average amount of from about 70 to about 250 IU FSH per day, and wherein the female subject is selected from the group consisting of
female subjects having an age of at least 35 years, preferably in the range of about 37 years to about 50 years;
female subjects having a serum level of anti-mullerian hormone (AMH) of 1.5
female subjects having an antral follicle count of 9 or less as the sum of both ovaries, preferably in the range of 4 to 8;
female subjects having a body mass index (BMI) of at lest 25 kg/m², preferably in the range of about 28 kg/m² to about 45 kg/m²; and
female subjects having undergone a previous conventional FSH stimulation cycle wherein the development of less than 4 oocytes was induced.

6. The method of claim 1, wherein a dosage regimen is used in step (a) wherein the recombinant FSH preparation is administered in an amount in IU which is 75% or less, preferably 50% or less of the amount recommended for recombinant FSH preparations produced by CHO cells, in particular Gonal-f, in the same therapeutic situation.

7. The method of claim 1, wherein the method comprises only one cycle of controlled ovarian hyperstimulation.

8. The method of claim 1, wherein the oocytes are obtained in step (c) in the form of cumulus oocyte complexes (COCs), and/or wherein at least 4 of the oocytes obtained in step (c) are metaphase II oocytes.

9. The method of claim 1, wherein the method further comprises
(d) fertilizing at least one oocyte obtained in step (c); and
(e) transferring at least one fertilized oocyte or embryo derived therefrom into a female human patient.

10. The method of claim 9, wherein the method further comprises freezing or vitrificating at least one oocyte obtained in step (c) prior to step (d); or freezing or vitrificating at least one fertilized oocyte obtained in step (d) or at least one embryo derived therefrom prior to step (e).

11. The method of claim 9, wherein only a subset of the oocytes obtained in step (c) are fertilized in step (d), and/or wherein only a subset of the oocytes fertilized in step (d) are transferred into a female human patient in step (e), and wherein the oocytes not fertilized in step (d) or the fertilized oocytes or embryos not transferred into a female human patient in step (e) are optionally frozen or vitrificated for subsequent use.

12. The method of claim 1, wherein the administration of the recombinant FSH preparation in step (a) does not comprise the concurrent administration of another gonadotropin such as LH or hCG or another agent which induces or enhances follicle growth.

13. The method of claim 1, wherein the administration of the recombinant FSH preparation in step (a) comprises the preceding and/or concurrent administration of a GnRH agonist or a GnRH antagonist.

14. The method of claim 1, wherein the female subject undergoes assisted reproductive technology (ART), in particular including in vitro fertilization (IVF), intracytoplasmic sperm injection (ICSI), gamete intrafallopian transfer (GIFT), zygote intrafallopian transfer (ZIFT), and/or embryo transfer.

15. The method of claim 1, obtainable by production in the human cell line GT-5s deposited under the accession number DSM ACC3078, or a cell line derived therefrom or a cell line homologous thereto.

16. The method of claim 1, wherein the glycosylation pattern comprises the following characteristics:
(i) a relative amount of glycans carrying bisecting N-acetylglucosamine (bisGlcNAc) in the range of from about 25% to about 50% of all glycans attached to the FSH in the preparation;
(ii) a relative amount of 2,6-coupled sialic acid in the range of from about 53% to about 80% of all sialic acid residues attached to the FSH in the preparation;
(iii) a relative amount of sulfated glycans of at least 3% of all glycans attached to the FSH in the preparation;
(iv) a relative amount of glycans carrying outer arm fucose of 5% or less of all glycans attached to the FSH in the preparation;
(v) a relative amount of glycans carrying core fucose of at least 30% of all glycans attached to the FSH in the preparation;
(vi) a relative amount of at least tetraantennary glycans of at least 16% of all glycans attached to the FSH in the preparation;
(vii) a relative amount of glycans carrying one or more sialic acid residues of at least 88% of all glycans attached to the FSH in the preparation; and
(viii) a Z number of at least 210.

17. A method for stimulating follicle maturation in a female subject, comprising
(a) inducing or enhancing follicle growth in a female subject by administering a recombinant FSH preparation; and
(b) subsequently triggering ovulation;
wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation;
wherein triggering ovulation in step (b) is commenced at least 48 h after termination of the administration of the recombinant FSH preparation in step (a).

18. The method of claim 17, wherein triggering ovulation in step (b) is commenced about 60 h to about 120 h, preferably about 72 h to about 96 h after termination of the administration of the recombinant FSH preparation in step (a).

19. The method of claim 17, wherein triggering ovulation in step (b) is performed by administering hCG or a derivative thereof.

20. A method for controlled ovarian hyperstimulation for stimulating the development of multiple ovarian follicles in a female subject, comprising (a) administering to a female subject a recombinant FSH preparation using a dosage regimen wherein the recombinant FSH preparation is administered in an amount in IU which is 80% or less of the amount recommended for recombinant FSH preparations produced by CHO cells in the same therapeutic situation;
(b) triggering ovulation when there are multiple follicles with a mean diameter equal to or greater than 12 mm and/or when there is at least one follicle with a diameter of at least 17 mm;
(c) obtaining multiple oocytes from the female subject, wherein on average at least 5% more oocytes per female subject are obtained compared to a similar treatment with the amount recommended for recombinant FSH preparations produced by CHO cells in the same therapeutic situation;
wherein the recombinant FSH in the preparation has a glycosylation pattern comprising the following characteristics:
(i) a relative amount of glycans carrying bisecting N-acetylglucosamine (bisGlcNAc) of at least 20% of all glycans attached to the FSH in the preparation; and
(ii) a relative amount of 2,6-coupled sialic acid of at least 40% of all sialic acid residues attached to the FSH in the preparation.

21. The method of claim 20, wherein a dosage regimen is used in step (a), wherein the recombinant FSH preparation is administered in an amount in IU which is 50% or less of the amount recommended for recombinant FSH preparations produced by CHO cells, in particular Gonal-f, in the same therapeutic situation.

22. The method of claim 20, wherein in step (c) on average at least 5% more metaphase II oocytes and/or at least 5% more cumulus oocyte complexes are obtained compared to a similar treatment with the amount recommended for recombinant FSH preparations produced by CHO cells, in particular Gonal-f, in the same therapeutic situation.


SEQUENCE LISTING (2 sequences)

SEQ ID NO: 1 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 111 aa, Homo sapiens
NSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE